(19) United States
(12) Patent Application Publication
(10) Pub. No.: US 2025/0000367 A1
(43) Pub. Date: Jan. 2, 2025

(54) THERMAL MEASUREMENT-BASED BODY HEAT GENERATION MODEL, AN APPARATUS APPLYING THE RESPECTIVE MODEL AND A METHOD FOR CREATING THE RESPECTIVE MODEL

(71) Applicant: OURA HEALTH OY, Oulu (FI)

(72) Inventor: Jouni HUOPANA, Oulunsalo (FI)

(21) Appl. No.: 18/343,805

(22) Filed: Jun. 29, 2023

(51) Int. Cl. A61B 5/01; A61B 5/00; A61B 5/11

(52) U.S. Cl. CPC A61B 5/01; A61B 5/11; A61B 5/6826; A61B 5/7203; A61B 2560/0252; A61B 2562/0219; A61B 2562/0271

(57) ABSTRACT

Methods, systems, apparatuses and computer program products are for creating a body heat generation model of a user. A wearable device, such as a ring, is worn by the user. The method includes measuring finger skin temperatures with the wearable device, with at least one temperature sensor, and thereafter with an inverse problem-solving principle, a model is generated. Inner heat flux originating from the user's tissues is one component, and outer heat flux including cooling factors created by ambient temperature around the user and movements made by the user are other components. Thereafter, a thermal cross-sectional 2-dimensional map is constructed, including the finger tissue temperatures using heat convection information of the human tissue, and the result is a body heat generation model of the user. From the obtained model, an indicator of health or wellbeing information of the user may be determined.

### FIELD OF TECHNOLOGY

The following relates to wearable devices and data processing, including body heat generation model creation based on thermal measurements of a user by the respective wearable device.

### BACKGROUND

Some wearable devices may be configured to collect data, such as physiological data, from a user of the wearable device. For example, the wearable device may comprise one or more optical components to collect physiological data from the user. Furthermore, the wearable device may comprise sensors detecting temperatures of the user's finger, in order to keep track of the body temperature fluctuations of the user during the day and during the night as well.

However, a detected temperature value measured from a surface of a user tissue as such is a discrete value, and it provides a rather limited amount of information, especially as related to thermal and physiological phenomena happening well inside the tissues of the user's body.

### BRIEF DESCRIPTION OF THE DRAWINGS

FIG. 1 illustrates an example of a system that supports techniques for creating a body heat generation model of a user based on thermal measurements of the user, using a wearable device in accordance with aspects of the present disclosure,

FIG. 2 illustrates an example of a system that supports techniques for creating a body heat generation model of a user based on thermal measurements of the user, using a wearable device in accordance with aspects of the present disclosure,

FIG. 3A shows an example of a two-dimensional temperature result illustration of a cross-section of a user finger,

FIG. 3B shows temperature values as a function of time for four temperature sensors, in an example of the invention,

FIG. 3C illustrates an example of an inner heat flux, in W/m, determined with the help of the created model,

FIG. 3D illustrates an example of an outer heat flux, in W/m, determined with the help of the created model,

FIG. 3E shows a temperature behavior graph in an embodiment of the invention,

FIG. 3F illustrates an example of a manner how the model according to the invention generates temperature values in the locations of each temperature sensor, as a function of tissue depth from the skin,

FIG. 3G illustrates yet another example of a two-dimensional temperature result illustration of a cross-section of a user finger,

FIG. 4 illustrates a block diagram of an apparatus that supports techniques for creating a body heat generation model of a user based on thermal measurements of the user, using a wearable device in accordance with aspects of the present disclosure,

FIG. 5 illustrates a block diagram of a wearable device manager that supports techniques for creating a body heat generation model of a user based on thermal measurements of the user, using a wearable device in accordance with aspects of the present disclosure,

FIG. 6 illustrates a diagram of a system comprising a device that supports techniques for creating a body heat generation model of a user based on thermal measurements of the user, using a wearable device in accordance with aspects of the present disclosure, and

FIG. 7 illustrates a flowchart showing a method that support techniques for creating a body heat generation model of a user based on thermal measurements of the user, using a wearable device in accordance with aspects of the present disclosure.

### DETAILED DESCRIPTION

A wearable device may be configured to collect physiological data from a user so that a user may monitor various aspects of their health. For example, the wearable device may collect heart rate data, sleep data, blood pressure data, and the like. In order to collect the physiological data from the user, the wearable device may include one or more optical sensors. For example, a wearable device may transmit light using one or more light-emitting components positioned at a first location of the wearable device, and the wearable device may receive the transmitted light using one or more photodetectors positioned at a second location of the wearable device. In some examples, the wearable device may be a ring, and the optical sensors (e.g., light-emitting components, photodetectors) may be positioned along the inside housing of the ring such that the light travels through or reflects off of one or more layers of the user's finger.

Furthermore, the wearable device may comprise at least one temperature sensor, also positioned along the inside housing of the ring such that the temperature sensors will be able to sense the skin temperature of the finger, where the ring is worn.

Temperature of the human body is an important parameter in human wellbeing monitoring because the temperature of the user body directly reveals if the user has fever or a slight temperature, e.g. with body temperature exceeding 37,0° C. for some people. However, every person has his/her own unique baseline temperature, and the temperature may fluctuate from that baseline temperature based on individual physiological and demographic factors, for instance. It should be taken into account also that a well functioning wearable device (such as e.g. a ring) will obtain the basal body temperature (the body temperature when the user is fully at rest) from the temperature measured from the skin of the finger with good accuracy. However, it can be taken into account that the size of the ring may just be a bit “wrong”, meaning that the ring remains a bit loose around the user's finger. This in turn may give false indications on the finger skin temperatures, as the gap between the ring and the finger skin attracts heat flow away from the finger skin surface. However, in an embodiment of the invention, the system may trigger some sort of an alarm or an indication or an informative message to the user that the size of the used ring is not correct for the particular user.

There is a need for obtaining accurate temperature information from a human user concerning the basal body temperature. The temperature information and its fluctuations over time reveal many physiological pieces of information, and health information and even a forecast of an incoming illness. Also general wellbeing status or “freshness” (meaning: How well has the user slept the previous night?) might be derived at least partly based on the basal body temperature. Furthermore, professional sports athletes and dedicated training enthusiasts and also regular people taking care of their wellbeing through exercise might have it useful to check on their body's situation, and recovery levels, and the body temperature will at least partly indicate the recovery status of the user e.g. after a hard training session. In this sense, there is a problem for determining or measuring the inner heat generation from the user's body more accurately than before with a wearable device. However, concerning temperature measurements performed on the surface of the skin in any part of the user body, the surrounding environment such as air movements, clothing (type of clothing and their tightness/looseness on the user body) and generally the external temperature where the user stays, will have an effect on the temperature measured on the user's skin.

The present invention solves this problem, and it provides more accurate temperature information of the user body via a body heat generation model based on thermal measurements made on the skin of the finger of the user. In other words, an internal heat generation from the user body is more accurately determined as a function of time, and from that data, the present invention is able to determine an indicator of health or wellbeing information of the user, and inform that indicator of information to the user via e.g. a wireless telecommunication device, such as a smartphone of the user.

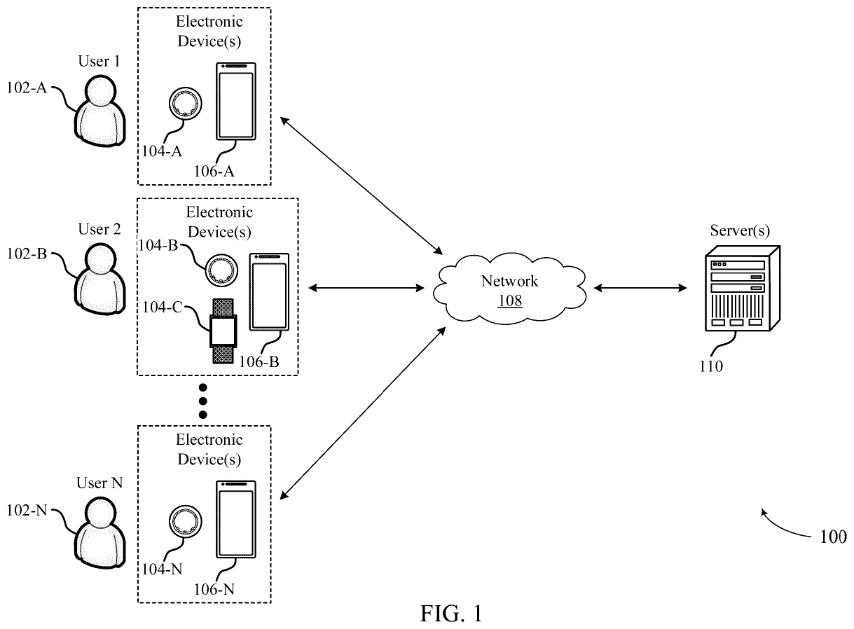

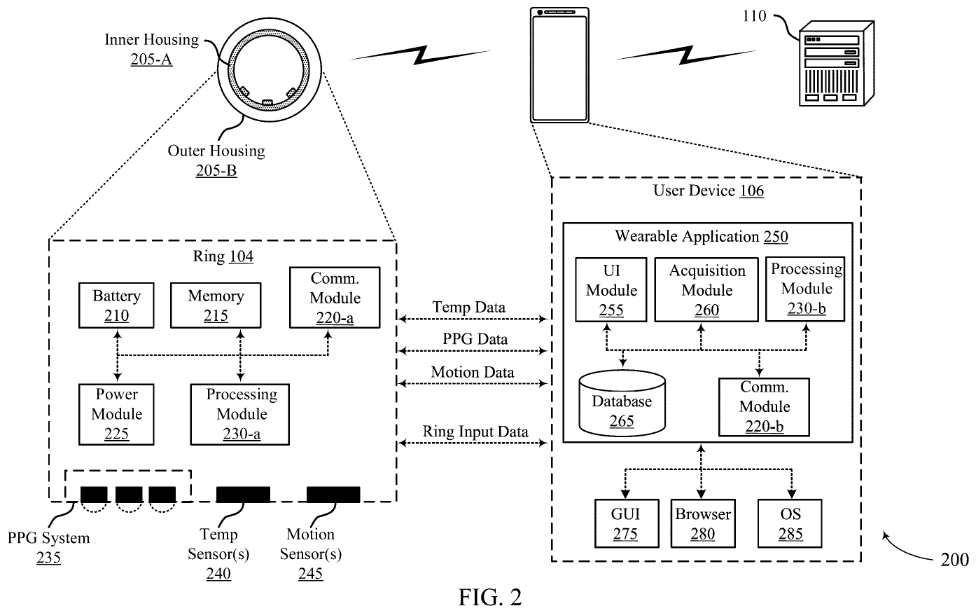

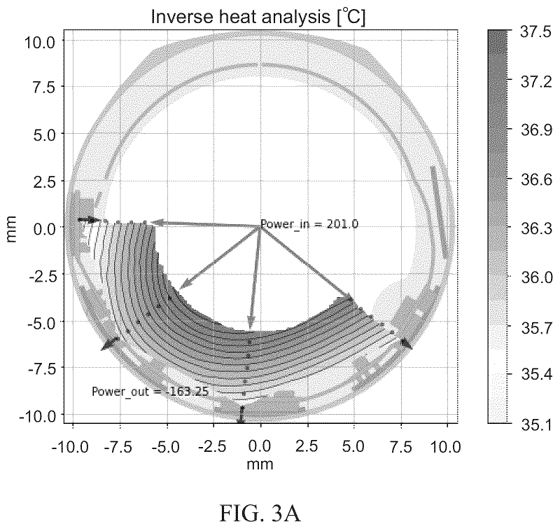

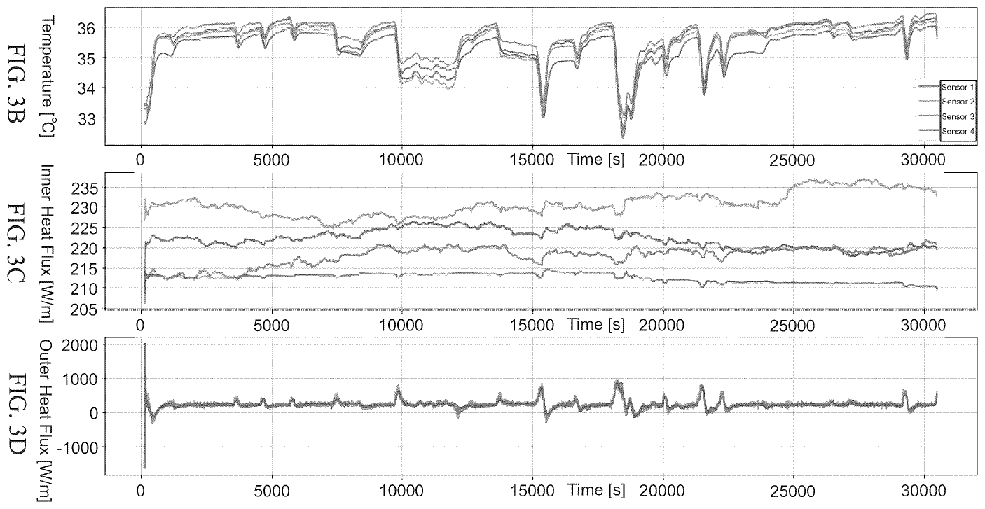

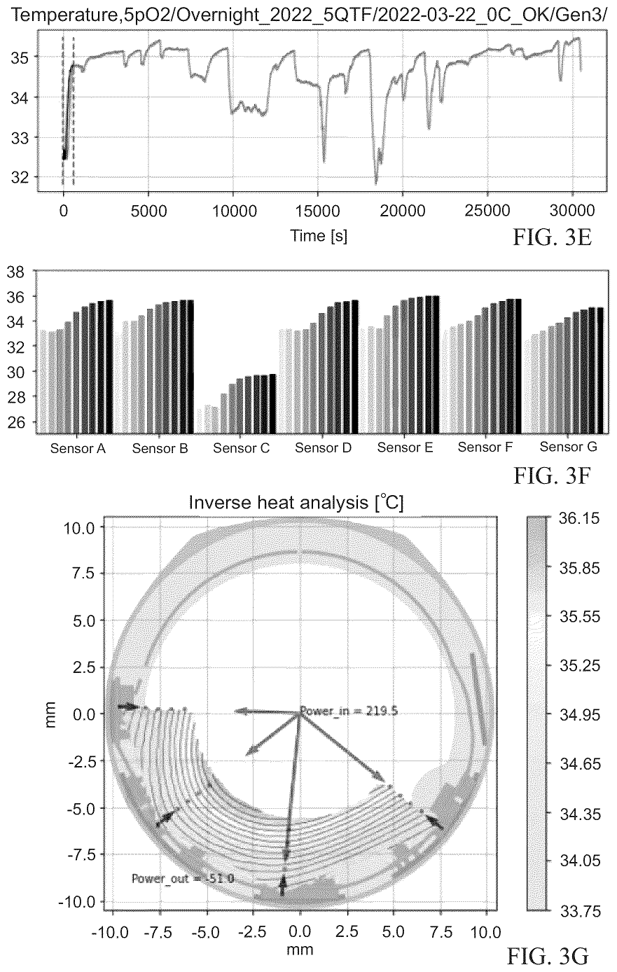

A more detailed description of the present invention, and its various embodiments, are discussed a bit later starting from FIG. 3. However, some general aspects concerning the applied system involving wearable devices and their users are discussed in connection with FIG. 1. Furthermore, some functional blocks and elements involved in the wearable device and in a wireless user device are discussed in connection with FIG. 2. All these matters can be embodied in connection with the inventive concept, in various different embodiments of the invention.

FIG. 1 illustrates an example of a system 100 that supports techniques for creating a body heat generation model of a user based on thermal measurements of the user, using a wearable device in accordance with aspects of the present disclosure. The system 100 includes a plurality of electronic devices (e.g., wearable devices 104, user devices 106) that may be worn and/or operated by one or more users 102. The system 100 further includes a network 108 and one or more servers 110.

The electronic devices may include any electronic devices known in the art, including wearable devices 104 (e.g., ring wearable devices, watch wearable devices, etc.), user devices 106 (e.g., smartphones, laptops, tablets). The electronic devices associated with the respective users 102 may include one or more of the following functionalities: 1) measuring physiological data, 2) storing the measured data, 3) processing the data, 4) providing outputs (e.g., via GUIs) to a user 102 based on the processed data, and 5) communicating data with one another and/or other computing devices. Different electronic devices may perform one or more of the functionalities.

Example wearable devices 104 may include wearable computing devices, such as a ring computing device (hereinafter “ring”) configured to be worn on a user's 102 finger, a wrist computing device (e.g., a smart watch, fitness band, or bracelet) configured to be worn on a user's 102 wrist, and/or a head mounted computing device (e.g., glasses/goggles). Wearable devices 104 may also include bands, straps (e.g., flexible or inflexible bands or straps), stick-on sensors, and the like, that may be positioned in other locations, such as bands around the head (e.g., a forehead headband), arm (e.g., a forearm band and/or bicep band), and/or leg (e.g., a thigh or calf band), behind the ear, under the armpit, and the like. Wearable devices 104 may also be attached to, or included in, articles of clothing. For example, wearable devices 104 may be included in pockets and/or pouches on clothing. As another example, wearable device 104 may be clipped and/or pinned to clothing, or it may otherwise be maintained within the vicinity of the user 102. Example articles of clothing may include, but are not limited to, hats, shirts, gloves, pants, socks, outerwear (e.g., jackets), and undergarments. In some implementations, wearable devices 104 may be included with other types of devices such as training/sporting devices that are used during physical activity. For example, wearable devices 104 may be attached to, or included in, a bicycle, skis, a tennis racket, a golf club, and/or training weights.

Much of the present disclosure may be described in the context of a ring wearable device 104. Accordingly, the terms “ring 104,” “wearable device 104,” and like terms, may be used interchangeably, unless noted otherwise herein. However, the use of the term “ring 104” is not to be regarded as limiting, as it is contemplated herein that aspects of the present disclosure may be performed using other wearable devices (e.g., watch wearable devices, necklace wearable device, bracelet wearable devices, earring wearable devices, anklet wearable devices, and the like).

In some aspects of the invention, user devices 106 may include handheld mobile computing devices, such as smartphones and tablet computing devices. User devices 106 may also include personal computers, such as laptop and desktop computing devices. Other example user devices 106 may include server computing devices that may communicate with other electronic devices (e.g., via the Internet). In some implementations, computing devices may include medical devices, such as external wearable computing devices (e.g., Holter monitors). Medical devices may also include implantable medical devices, such as pacemakers and cardioverter defibrillators. Other example user devices 106 may include home computing devices, such as internet of things (IoT) devices (e.g., IoT devices), smart televisions, smart speakers, smart displays (e.g., video call displays), hubs (e.g., wireless communication hubs), security systems, smart appliances (e.g., thermostats and refrigerators), and fitness equipment.

Some electronic devices (e.g., wearable devices 104, user devices 106) may measure physiological parameters of respective users 102, such as photoplethysmography waveforms, continuous skin temperature, a pulse waveform, respiration rate, heart rate, heart rate variability (HRV), actigraphy, galvanic skin response, pulse oximetry, and/or other physiological parameters. Some electronic devices that measure physiological parameters may also perform some/all of the calculations described herein. Some electronic devices may not measure physiological parameters but may perform some/all of the calculations described herein. For example, a ring (e.g., wearable device 104), mobile device application, or a server computing device may process received physiological data that was measured by other devices.

In some implementations, a user 102 may operate, or may be associated with, multiple electronic devices, some of which may measure physiological parameters and some of which may process the measured physiological parameters. In some implementations, a user 102 may have a ring (e.g., wearable device 104) that measures physiological parameters. The user 102 may also have, or be associated with, a user device 106 (e.g., mobile device, smartphone), where the wearable device 104 and the user device 106 are communicatively coupled to one another. In some cases, the user device 106 may receive data from the wearable device 104 and perform some or all of the calculations described herein. In some implementations, the user device 106 may also measure physiological parameters described herein, such as motion/activity parameters.

For example, as illustrated in FIG. 1, a first user 102-a (User 1) may operate, or may be associated with, a wearable device 104-a (e.g., ring 104-a) and a user device 106-a that may operate as described herein. In this example, the user device 106-a associated with user 102-a may process/store physiological parameters measured by the ring 104-a. Comparatively, a second user 102-b (User 2) may be associated with a ring 104-b, a watch wearable device 104-c (e.g., watch 104-c), and a user device 106-b, where the user device 106-b associated with user 102-b may process/store physiological parameters measured by the ring 104-b and/or the watch 104-c. Moreover, an n^{th} user 102-n (User N) may be associated with an arrangement of electronic devices described herein (e.g., ring 104-n, user device 106-n). In some aspects of the invention, wearable devices 104 (e.g., rings 104, watches 104) and other electronic devices may be communicatively coupled to the user devices 106 of the respective users 102 via Bluetooth, Wi-Fi, and other wireless protocols.

In some implementations, the rings 104 (e.g., wearable devices 104) of the system 100 may be configured to collect physiological data from the respective users 102 based on arterial blood flow within the user's finger. In particular, a ring 104 may utilize one or more light-emitting components, such as LEDs (e.g., red LEDs, green LEDs) that emit light on the palm-side of a user's finger to collect physiological data based on arterial blood flow within the user's finger. In general, the terms light-emitting components, light-emitting elements, and like terms, may include, but are not limited to, LEDs, micro-LEDs, mini-LEDs, laser diodes (LDs), and the like.

In some cases, the system 100 may be configured to collect physiological data from the respective users 102 based on blood flow diffused into a microvascular bed of skin with capillaries and arterioles. For example, the system 100 may collect PPG data based on a measured amount of blood diffused into the microvascular system of capillaries and arterioles. In some implementations, the ring 104 may acquire the physiological data using a combination of both green and red LEDs. The physiological data may include any physiological data known in the art including, but not limited to, temperature data, accelerometer data (e.g., movement/motion data), heart rate data, HRV data, blood oxygen level data, or any combination thereof.

The use of both green and red LEDs may provide several advantages over other solutions, as red and green LEDs have been found to have their own distinct advantages when acquiring physiological data under different conditions (e.g., light/dark, active/inactive) and via different parts of the body, and the like. For example, green LEDs have been found to exhibit better performance during exercise. Moreover, using multiple LEDs (e.g., green and red LEDs) distributed around the ring 104 has been found to exhibit superior performance as compared to wearable devices that utilize LEDs that are positioned close to one another, such as within a watch wearable device. Furthermore, the blood vessels in the finger (e.g., arteries, capillaries) are more accessible via LEDs as compared to blood vessels in the wrist. In particular, arteries in the wrist are positioned on the bottom of the wrist (e.g., palm-side of the wrist), meaning only capillaries are accessible on the top of the wrist (e.g., back of hand side of the wrist), where wearable watch devices and similar devices are typically worn. As such, utilizing LEDs and other sensors within a ring 104 has been found to exhibit superior performance as compared to wearable devices worn on the wrist, as the ring 104 may have greater access to arteries (as compared to capillaries), thereby resulting in stronger signals and more valuable physiological data.

The electronic devices of the system 100 (e.g., user devices 106, wearable devices 104) may be communicatively coupled to one or more servers 110 via wired or wireless communication protocols. For example, as shown in FIG. 1, the electronic devices (e.g., user devices 106) may be communicatively coupled to one or more servers 110 via a network 108. The network 108 may implement transfer control protocol and internet protocol (TCP/IP), such as the Internet, or may implement other network 108 protocols. Network connections between the network 108 and the respective electronic devices may facilitate transport of data via email, web, text messages, mail, or any other appropriate form of interaction within a computer network 108. For example, in some implementations, the ring 104-a associated with the first user 102-a may be communicatively coupled to the user device 106-a, where the user device 106-a is communicatively coupled to the servers 110 via the network 108. In additional or alternative cases, wearable devices 104 (e.g., rings 104, watches 104) may be directly communicatively coupled to the network 108.

The system 100 may offer an on-demand database service between the user devices 106 and the one or more servers 110. In some cases, the servers 110 may receive data from the user devices 106 via the network 108, and they may store and analyze the data. Similarly, the servers 110 may provide data to the user devices 106 via the network 108. In some cases, the servers 110 may be located at one or more data centers. The servers 110 may be used for data storage, management, and processing. In some implementations, the servers 110 may provide a web-based interface to the user device 106 via web browsers.

In some aspects of the invention, the system 100 may detect periods of time that a user 102 is asleep and classify periods of time that the user 102 is asleep into one or more sleep stages (e.g., sleep stage classification). For example, as shown in FIG. 1, User 102-a may be associated with a wearable device 104-a (e.g., ring 104-a) and a user device 106-a. In this example, the ring 104-a may collect physiological data associated with the user 102-a, including temperature, heart rate, HRV, respiratory rate, and the like. In some aspects of the invention, data collected by the ring 104-a may be input to a machine learning classifier, where the machine learning classifier is configured to determine periods of time that the user 102-a is (or was) asleep. Moreover, the machine learning classifier may be configured to classify periods of time into different sleep stages, including an awake sleep stage, a rapid eye movement (REM) sleep stage, a light sleep stage (non-REM (NREM)), and a deep sleep stage (NREM). In some aspects of the invention, the classified sleep stages may be displayed to the user 102-a via a GUI of the user device 106-a. Sleep stage classification may be used to provide feedback to a user 102-a regarding the user's sleeping patterns, such as recommended bedtimes, recommended wake-up times, and the like. Moreover, in some implementations, sleep stage classification techniques described herein may be used to calculate scores for the respective user, such as Sleep Scores, Readiness Scores, and the like.

In some aspects of the invention, the system 100 may utilize circadian rhythm-derived features to further improve physiological data collection, data processing procedures, and other techniques described herein. The term circadian rhythm may refer to a natural, internal process that regulates an individual's sleep-wake cycle, that repeats approximately every 24 hours. In this regard, techniques described herein may utilize circadian rhythm adjustment models to improve physiological data collection, analysis, and data processing. For example, a circadian rhythm adjustment model may be input into a machine learning classifier along with physiological data collected from the user 102-a via the wearable device 104-a. In this example, the circadian rhythm adjustment model may be configured to “weight,” or adjust, physiological data collected throughout a user's natural, approximately 24-hour circadian rhythm. In some implementations, the system may initially start with a “baseline” circadian rhythm adjustment model, and it may modify the baseline model using physiological data collected from each user 102 to generate tailored, individualized circadian rhythm adjustment models that are specific to each respective user 102.

In some aspects of the invention, the system 100 may utilize other biological rhythms to further improve physiological data collection, analysis, and processing by phase of these other rhythms. For example, if a weekly rhythm is detected within an individual's baseline data, then the model may be configured to adjust “weights” of data by day of the week. Biological rhythms that may require adjustment to the model by this method include: 1) ultradian (faster than a day rhythms, including sleep cycles in a sleep state, and oscillations from less than an hour to several hours periodicity in the measured physiological variables during wake state; 2) circadian rhythms; 3) non-endogenous daily rhythms shown to be imposed on top of circadian rhythms, as in work schedules; 4) weekly rhythms, or other artificial time periodicities exogenously imposed (e.g., in a hypothetical culture with 12 day “weeks,” 12 day rhythms could be used); 5) multi-day ovarian rhythms in women and spermatogenesis rhythms in men; 6) lunar rhythms (relevant for individuals living with low or no artificial lights); and 7) seasonal rhythms.

The biological rhythms are not always stationary rhythms. For example, many women experience variability in ovarian cycle length across cycles, and ultradian rhythms are not expected to occur at exactly the same time or periodicity across days even within a user. As such, signal processing techniques sufficient to quantify the frequency composition while preserving temporal resolution of these rhythms in physiological data may be used to improve detection of these rhythms, to assign phase of each rhythm to each moment in time measured, and to thereby modify adjustment models and comparisons of time intervals. The biological rhythm-adjustment models and parameters can be added in linear or non-linear combinations as appropriate to more accurately capture the dynamic physiological baselines of an individual or a group of individuals.

FIG. 2 illustrates an example of a system 200 that supports techniques for creating a body heat generation model of a user based on thermal measurements of the user, using a wearable device in accordance with aspects of the present disclosure. The system 200 may implement, or be implemented by, system 100. In particular, system 200 illustrates an example of a ring 104 (e.g., wearable device 104), a user device 106, and a server 110, as described with reference to FIG. 1.

In some aspects of the invention, the ring 104 may be configured to be worn around a user's finger, and it may determine one or more user physiological parameters when worn around the user's finger. Example measurements and determinations may include, but are not limited to, user skin temperature, pulse waveforms, respiratory rate, heart rate, HRV, blood oxygen levels, and the like.

The system 200 further includes a user device 106 (e.g., a smartphone) in communication with the ring 104. For example, the ring 104 may be in wireless and/or wired communication with the user device 106. In some implementations, the ring 104 may send measured and processed data (e.g., temperature data, photoplethysmogram (PPG) data, motion/accelerometer data, ring input data, and the like) to the user device 106. The user device 106 may also send data to the ring 104, such as ring 104 firmware/configuration updates. The user device 106 may process data. In some implementations, the user device 106 may transmit data to the server 110 for processing and/or storage.

The ring 104 may include a housing 205 that may include an inner housing 205-a and an outer housing 205-b. In some aspects of the invention, the housing 205 of the ring 104 may store or otherwise include various components of the ring including, but not limited to, device electronics, a power source (e.g., battery 210, and/or capacitor), one or more substrates (e.g., printable circuit boards) that interconnect the device electronics and/or power source, and the like. The device electronics may include device modules (e.g., hardware/software), such as: a processing module 230-a, a memory 215, a communication module 220-a, a power module 225, and the like. The device electronics may also include one or more sensors. Example sensors may include one or more temperature sensors 240, a PPG sensor assembly (e.g., PPG system 235), and one or more motion sensors 245.

The sensors may include associated modules (not illustrated) configured to communicate with the respective components/modules of the ring 104, and generate signals associated with the respective sensors. In some aspects of the invention, each of the components/modules of the ring 104 may be communicatively coupled to one another via wired or wireless connections. Moreover, the ring 104 may include additional and/or alternative sensors or other components that are configured to collect physiological data from the user, including light sensors (e.g., LEDs), oximeters, and the like.

The ring 104 shown and described with reference to FIG. 2 is provided solely for illustrative purposes. As such, the ring 104 may include additional or alternative components as those illustrated in FIG. 2. Other rings 104 that provide functionality described herein may be fabricated. For example, rings 104 with fewer components (e.g., sensors) may be fabricated. In a specific example, a ring 104 with a single temperature sensor 240 (or other sensor), a power source, and device electronics configured to read the single temperature sensor 240 (or other sensor) may be fabricated. In another specific example, a temperature sensor 240 (or other sensor) may be attached to a user's finger (e.g., using clamps, spring loaded clamps, etc.). In this case, the sensor may be wired to another computing device, such as a wrist worn computing device that reads the temperature sensor 240 (or other sensor). In other examples, a ring 104 that includes additional sensors and processing functionality may be fabricated.

The housing 205 may include one or more housing 205 components. The housing 205 may include an outer housing 205-b component (e.g., a shell) and an inner housing 205-a component (e.g., a molding). The housing 205 may include additional components (e.g., additional layers) not explicitly illustrated in FIG. 2. For example, in some implementations, the ring 104 may include one or more insulating layers that electrically insulate the device electronics and other conductive materials (e.g., electrical traces) from the outer housing 205-b (e.g., a metal outer housing 205-b). The housing 205 may provide structural support for the device electronics, battery 210, substrate(s), and other components. For example, the housing 205 may protect the device electronics, battery 210, and substrate(s) from mechanical forces, such as pressure and impacts. The housing 205 may also protect the device electronics, battery 210, and substrate(s) from water and/or other chemicals.

The outer housing 205-b may be fabricated from one or more materials. In some implementations, the outer housing 205-b may include a metal, such as titanium, that may provide strength and abrasion resistance at a relatively light weight. The outer housing 205-b may also be fabricated from other materials, such polymers. In some implementations, the outer housing 205-b may be protective as well as decorative.

The inner housing 205-a may be configured to interface with the user's finger. The inner housing 205-a may be formed from a polymer (e.g., a medical grade polymer) or other material. In some implementations, the inner housing 205-a may be transparent. For example, the inner housing 205-a may be transparent to light emitted by the PPG light emitting diodes (LEDs). In some implementations, the inner housing 205-a component may be molded onto the outer housing 205-b. For example, the inner housing 205-a may include a polymer that is molded (e.g., injection molded) to fit into an outer housing 205-b metallic shell.

The ring 104 may include one or more substrates (not illustrated). The device electronics and battery 210 may be included on the one or more substrates. For example, the device electronics and battery 210 may be mounted on one or more substrates. Example substrates may include one or more printed circuit boards (PCBs), such as flexible PCB (e.g., polyimide). In some implementations, the electronics/battery 210 may include surface mounted devices (e.g., surface-mount technology (SMT) devices) on a flexible PCB. In some implementations, the one or more substrates (e.g., one or more flexible PCBs) may include electrical traces that provide electrical communication between device electronics. The electrical traces may also connect the battery 210 to the device electronics.

The device electronics, battery 210, and substrates may be arranged in the ring 104 in a variety of ways. In some implementations, one substrate that includes device electronics may be mounted along the bottom of the ring 104 (e.g., the bottom half), such that the sensors (e.g., PPG system 235, temperature sensors 240, motion sensors 245, and other sensors) interface with the underside of the user's finger. In these implementations, the battery 210 may be included along the top portion of the ring 104 (e.g., on another substrate).

The various components/modules of the ring 104 represent functionality (e.g., circuits and other components) that may be included in the ring 104. Modules may include any discrete and/or integrated electronic circuit components that implement analog and/or digital circuits capable of producing the functions attributed to the modules herein. For example, the modules may include analog circuits (e.g., amplification circuits, filtering circuits, analog/digital conversion circuits, and/or other signal conditioning circuits). The modules may also include digital circuits (e.g., combinatorial or sequential logic circuits, memory circuits etc.).

The memory 215 (memory module) of the ring 104 may include any volatile, non-volatile, magnetic, or electrical media, such as a random-access memory (RAM), read-only memory (ROM), non-volatile RAM (NVRAM), electrically erasable programmable ROM (EEPROM), flash memory, or any other memory device. The memory 215 may store any of the data described herein. For example, the memory 215 may be configured to store data (e.g., motion data, temperature data, PPG data) collected by the respective sensors and PPG system 235. Furthermore, memory 215 may include instructions that, when executed by one or more processing circuits, cause the modules to perform various functions attributed to the modules herein. The device electronics of the ring 104 described herein are only example device electronics. As such, the types of electronic components used to implement the device electronics may vary based on design considerations.

The functions attributed to the modules of the ring 104 described herein may be embodied as one or more processors, hardware, firmware, software, or any combination thereof. Depiction of different features as modules is intended to highlight different functional aspects and does not necessarily imply that such modules must be realized by separate hardware/software components. Rather, functionality associated with one or more modules may be performed by separate hardware/software components or integrated within common hardware/software components.

The processing module 230-a of the ring 104 may include one or more processors (e.g., processing units), microcontrollers, digital signal processors, systems on a chip (SOCs), and/or other processing devices. The processing module 230-a communicates with the modules included in the ring 104. For example, the processing module 230-a may transmit/receive data to/from the modules and other components of the ring 104, such as the sensors. As described herein, the modules may be implemented by various circuit components. Accordingly, the modules may also be referred to as circuits (e.g., a communication circuit and power circuit).

The processing module 230-a may communicate with the memory 215. The memory 215 may include computer-readable instructions that, when executed by the processing module 230-a, cause the processing module 230-a to perform the various functions attributed to the processing module 230-a herein. In some implementations, the processing module 230-a (e.g., a microcontroller) may include additional features associated with other modules, such as communication functionality provided by the communication module 220-a (e.g., an integrated Bluetooth Low Energy transceiver) and/or additional onboard memory 215.

The communication module 220-a may include circuits that provide wireless and/or wired communication with the user device 106 (e.g., communication module 220-b of the user device 106). In some implementations, the communication modules 220-a, 220-b may include wireless communication circuits, such as Bluetooth circuits and/or Wi-Fi circuits. In some implementations, the communication modules 220-a, 220-b may include wired communication circuits, such as Universal Serial Bus (USB) communication circuits. Using the communication module 220-a, the ring 104 and the user device 106 may be configured to communicate with each other. The processing module 230-a of the ring may be configured to transmit/receive data to/from the user device 106 via the communication module 220-a. Example data may comprise, but is not limited to, motion data, temperature data, pulse waveforms, heart rate data, HRV data, PPG data, and status updates (e.g., charging status, battery charge level, and/or ring 104 configuration settings). The processing module 230-a of the ring may also be configured to receive updates (e.g., software/firmware updates) and data from the user device 106.

The ring 104 may include a battery 210 (e.g., a rechargeable battery 210). An example battery 210 may include a Lithium-Ion or Lithium-Polymer type battery 210, although a variety of battery 210 options are possible. The battery 210 may be wirelessly charged. In some implementations, the ring 104 may include a power source other than the battery 210, such as a capacitor. The power source (e.g., battery 210 or capacitor) may have a curved geometry that matches the curve of the ring 104. In some aspects of the invention, a charger or other power source may include additional sensors that may be used to collect data in addition to, or that supplements, data collected by the ring 104 itself. Moreover, a charger or other power source for the ring 104 may function as a user device 106, in which case the charger or other power source for the ring 104 may be configured to receive data from the ring 104, store and/or process data received from the ring 104 and communicate data between the ring 104 and the servers 110.

In some aspects of the invention, the ring 104 includes a power module 225 that may control charging of the battery 210. For example, the power module 225 may interface with an external wireless charger that charges the battery 210 when interfaced with the ring 104. The charger may include a datum structure that mates with a ring 104 datum structure to create a specified orientation with the ring 104 during charging. The power module 225 may also regulate voltage(s) of the device electronics, regulate power output to the device electronics, and monitor the state of charge of the battery 210. In some implementations, the battery 210 may include a protection circuit module (PCM) that protects the battery 210 from high current discharge, over voltage during charging, and under voltage during discharge. The power module 225 may also include electro-static discharge (ESD) protection.

The one or more temperature sensors 240 may be electrically coupled to the processing module 230-a. The temperature sensor 240 may be configured to generate a temperature signal (e.g., temperature data) that indicates a temperature read or sensed by the temperature sensor 240. The processing module 230-a may determine a temperature of the user in the location of the temperature sensor 240. For example, in the ring 104, temperature data generated by the temperature sensor 240 may indicate a temperature of a user at the user's finger (e.g., skin temperature). In some implementations, the temperature sensor 240 may contact the user's skin. In other implementations, a portion of the housing 205 (e.g., the inner housing 205-a) may form a barrier (e.g., a thin, thermally conductive barrier) between the temperature sensor 240 and the user's skin. In some implementations, portions of the ring 104 configured to contact the user's finger may have thermally conductive portions and thermally insulative portions. The thermally conductive portions may conduct heat from the user's finger to the temperature sensors 240. The thermally insulative portions may insulate portions of the ring 104 (e.g., the temperature sensor 240) from ambient temperature.

In some implementations, the temperature sensor 240 may generate a digital signal (e.g., temperature data) that the processing module 230-a may use to determine the temperature. As another example, in cases where the temperature sensor 240 includes a passive sensor, the processing module 230-a (or a temperature sensor 240 module) may measure a current/voltage generated by the temperature sensor 240 and determine the temperature based on the measured current/voltage. Example temperature sensors 240 may comprise a thermistor, such as a negative temperature coefficient (NTC) thermistor, or other types of sensors including resistors, transistors, diodes, and/or other electrical/electronic components.

The processing module 230-a may sample the user's temperature over time. For example, the processing module 230-a may sample the user's temperature according to a sampling rate. An example sampling rate may include one sample per second, although the processing module 230-a may be configured to sample the temperature signal at other sampling rates that are higher or lower than one sample per second. In some implementations, the processing module 230-a may sample the user's temperature continuously throughout the day and night. Sampling at a sufficient rate (e.g., one sample per second) throughout the day may provide sufficient temperature data for analysis described herein.

The processing module 230-a may store the sampled temperature data in memory 215. In some implementations, the processing module 230-a may process the sampled temperature data. For example, the processing module 230-a may determine average temperature values over a period of time. In one example, the processing module 230-a may determine an average temperature value each minute by summing all temperature values collected over the minute and dividing by the number of samples over the minute. In a specific example where the temperature is sampled at one sample per second, the average temperature may be a sum of all sampled temperatures for one minute divided by sixty seconds. The memory 215 may store the average temperature values over time. In some implementations, the memory 215 may store average temperatures (e.g., one per minute) instead of sampled temperatures in order to conserve memory 215.

The sampling rate, which may be stored in memory 215, may be configurable. In some implementations, the sampling rate may be the same throughout the day and night. In other implementations, the sampling rate may be changed throughout the day/night. In some implementations, the ring 104 may filter/reject temperature readings, such as large spikes in temperature that are not indicative of physiological changes (e.g., a temperature spike from a hot shower). In some implementations, the ring 104 may filter/reject temperature readings that may not be reliable due to other factors, such as excessive motion during exercise (e.g., as indicated by a motion sensor 245).

The ring 104 (e.g., communication module) may transmit the sampled and/or average temperature data to the user device 106 for storage and/or further processing. The user device 106 may transfer the sampled and/or average temperature data to the server 110 for storage and/or further processing.

Although the ring 104 is illustrated as including a single temperature sensor 240, the ring 104 may include multiple temperature sensors 240 in one or more locations, such as arranged along the inner housing 205-a near the user's finger. In some implementations, the temperature sensors 240 may be stand-alone temperature sensors 240. Additionally, or alternatively, one or more temperature sensors 240 may be included with other components (e.g., packaged with other components), such as with the accelerometer and/or the processor.

The processing module 230-a may acquire and process data from multiple temperature sensors 240 in a similar manner described with respect to a single temperature sensor 240. For example, the processing module 230 may individually sample, average, and store temperature data from each of the multiple temperature sensors 240. In other examples, the processing module 230-a may sample the sensors at different rates and average/store different values for the different sensors. In some implementations, the processing module 230-a may be configured to determine a single temperature based on the average of two or more temperatures determined by two or more temperature sensors 240 in different locations on the finger.

The temperature sensors 240 on the ring 104 may acquire distal temperatures at the user's finger (e.g., any finger). For example, one or more temperature sensors 240 on the ring 104 may acquire a user's temperature from the underside of a finger or at a different location on the finger. In some implementations, the ring 104 may continuously acquire distal temperature (e.g., at a sampling rate). Although distal temperature measured by a ring 104 at the finger is described herein, other devices may measure temperature at the same/different locations. In some cases, the distal temperature measured at a user's finger may differ from the temperature measured at a user's wrist or other external body location. Additionally, the distal temperature measured at a user's finger (e.g., a “shell” temperature) may differ from the user's core temperature. As such, the ring 104 may provide a useful temperature signal that may not be acquired at other internal/external locations of the body. In some cases, continuous temperature measurement at the finger may capture temperature fluctuations (e.g., small or large fluctuations) that may not be evident in the core temperature. For example, continuous temperature measurement at the finger may capture minute-to-minute or hour-to-hour temperature fluctuations that provide additional insight that may not be provided by other temperature measurements elsewhere in the body.

The ring 104 may comprise a PPG system 235. The PPG system 235 may comprise one or more optical transmitters that transmit light. The PPG system 235 may also comprise one or more optical receivers that receive light transmitted by the one or more optical transmitters. An optical receiver may generate a signal (hereinafter “PPG” signal) that indicates an amount of light received by the optical receiver. The optical transmitters may illuminate a region of the user's finger. The PPG signal generated by the PPG system 235 may indicate the perfusion of blood in the illuminated region. For example, the PPG signal may indicate blood volume changes in the illuminated region caused by a user's pulse pressure. The processing module 230-a may sample the PPG signal and determine a user's pulse waveform based on the PPG signal. The processing module 230-a may determine a variety of physiological parameters based on the user's pulse waveform, such as a user's respiratory rate, heart rate, HRV, oxygen saturation, and other circulatory parameters.

In some implementations, the PPG system 235 may be configured as a reflective PPG system 235 where the optical receiver(s) receive transmitted light that is reflected through the region of the user's finger. In some implementations, the PPG system 235 may be configured as a transmissive PPG system 235 where the optical transmitter(s) and optical receiver(s) are arranged opposite to one another, such that light is transmitted directly through a portion of the user's finger to the optical receiver(s).

The number and ratio of transmitters and receivers included in the PPG system 235 may vary. Example optical transmitters may comprise light-emitting diodes (LEDs). The optical transmitters may transmit light in the infrared spectrum and/or other spectrums. Example optical receivers may comprise, but are not limited to, photosensors, phototransistors, and photodiodes. The optical receivers may be configured to generate PPG signals in response to the wavelengths received from the optical transmitters. The location of the transmitters and receivers may vary. Additionally, a single device may comprise reflective and/or transmissive PPG systems 235.

The PPG system 235 illustrated in FIG. 2 may comprise a reflective PPG system 235 in some implementations. In these implementations, the PPG system 235 may comprise a centrally located optical receiver (e.g., at the bottom of the ring 104) and two optical transmitters located on each side of the optical receiver. In this implementation, the PPG system 235 (e.g., optical receiver) may generate the PPG signal based on light received from one or both of the optical transmitters. In other implementations, other placements, combinations, and/or configurations of one or more optical transmitters and/or optical receivers are contemplated.

The processing module 230-a may control one or both of the optical transmitters to transmit light while sampling the PPG signal generated by the optical receiver. In some implementations, the processing module 230-a may cause the optical transmitter with the stronger received signal to transmit light while sampling the PPG signal generated by the optical receiver. For example, the selected optical transmitter may continuously emit light while the PPG signal is sampled at a sampling rate (e.g., 250 Hz).

Sampling the PPG signal generated by the PPG system 235 may result in a pulse waveform that may be referred to as a “PPG.” The pulse waveform may indicate blood pressure vs time for multiple cardiac cycles. The pulse waveform may comprise peaks that indicate cardiac cycles. Additionally, the pulse waveform may comprise respiratory induced variations that may be used to determine respiration rate. The processing module 230-a may store the pulse waveform in memory 215 in some implementations. The processing module 230-a may process the pulse waveform as it is generated and/or from memory 215 to determine user physiological parameters described herein.

The processing module 230-a may determine the user's heart rate based on the pulse waveform. For example, the processing module 230-a may determine heart rate (e.g., in beats per minute) based on the time between peaks in the pulse waveform. The time between peaks may be referred to as an inter beat interval (IBI). The processing module 230-a may store the determined heart rate values and IBI values in memory 215.

The processing module 230-a may determine HRV over time. For example, the processing module 230-a may determine HRV based on the variation in the IBIs. The processing module 230-a may store the HRV values over time in the memory 215. Moreover, the processing module 230-a may determine the user's respiratory rate over time. For example, the processing module 230-a may determine respiratory rate based on frequency modulation, amplitude modulation, or baseline modulation of the user's IBI values over a period of time. Respiratory rate may be calculated in breaths per minute or as another breathing rate (e.g., breaths per 30 seconds). The processing module 230-a may store user respiratory rate values over time in the memory 215.

The ring 104 may comprise one or more motion sensors 245, such as one or more accelerometers (e.g., 6-D accelerometers) and/or one or more gyroscopes (gyros). The motion sensors 245 may generate motion signals that indicate motion of the sensors. For example, the ring 104 may comprise one or more accelerometers that generate acceleration signals that indicate acceleration of the accelerometers. As another example, the ring 104 may comprise one or more gyro sensors that generate gyro signals that indicate angular motion (e.g., angular velocity) and/or changes in orientation. The motion sensors 245 may be comprised in one or more sensor packages. An example accelerometer/gyro sensor is a Bosch BM1160 inertial micro electro-mechanical system (MEMS) sensor that may measure angular rates and accelerations in three perpendicular axes.

The processing module 230-a may sample the motion signals at a sampling rate (e.g., 50 Hz) and determine the motion of the ring 104 based on the sampled motion signals. For example, the processing module 230-a may sample acceleration signals to determine acceleration of the ring 104. As another example, the processing module 230-a may sample a gyro signal to determine angular motion. In some implementations, the processing module 230-a may store motion data in memory 215. Motion data may comprise sampled motion data as well as motion data that is calculated based on the sampled motion signals (e.g., acceleration and angular values).

The ring 104 may store a variety of data described herein. For example, the ring 104 may store temperature data, such as raw sampled temperature data and calculated temperature data (e.g., average temperatures). As another example, the ring 104 may store PPG signal data, such as pulse waveforms and data calculated based on the pulse waveforms (e.g., heart rate values, IBI values, HRV values, and respiratory rate values). The ring 104 may also store motion data, such as sampled motion data that indicates linear and angular motion.

The ring 104, or other computing device, may calculate and store additional values based on the sampled/calculated physiological data. For example, the processing module 230 may calculate and store various metrics, such as sleep metrics (e.g., a Sleep Score), activity metrics, and readiness metrics. In some implementations, additional values/metrics may be referred to as “derived values.” The ring 104, or other computing/wearable device, may calculate a variety of values/metrics with respect to motion. Example derived values for motion data may comprise, but are not limited to, motion count values, regularity values, intensity values, metabolic equivalence of task values (METs), and orientation values. Motion counts, regularity values, intensity values, and METs may indicate an amount of user motion (e.g., velocity/acceleration) over time. Orientation values may indicate how the ring 104 is oriented on the user's finger and if the ring 104 is worn on the left hand or on the right hand.

In some implementations, motion counts and regularity values may be determined by counting a number of acceleration peaks within one or more periods of time (e.g., one or more 30 second to 1 minute periods). Intensity values may indicate a number of movements and the associated intensity (e.g., acceleration values) of the movements. The intensity values may be categorized as low, medium, and high, depending on associated threshold acceleration values. METs may be determined based on the intensity of movements during a period of time (e.g., 30 seconds), the regularity/irregularity of the movements, and the number of movements associated with the different intensities.

In some implementations, the processing module 230-a may compress the data stored in memory 215. For example, the processing module 230-a may delete sampled data after making calculations based on the sampled data. As another example, the processing module 230-a may average data over longer periods of time in order to reduce the number of stored values. In a specific example, if average temperatures for a user over one minute are stored in memory 215, the processing module 230-a may calculate average temperatures over a five-minute time period for storage, and then subsequently erase the one-minute average temperature data. The processing module 230-a may compress data based on a variety of factors, such as the total amount of used/available memory 215 and/or an elapsed time since the ring 104 last transmitted the data to the user device 106.

Although a user's physiological parameters may be measured by sensors comprised on a ring 104, other devices may measure a user's physiological parameters. For example, although a user's temperature may be measured by a temperature sensor 240 comprised in a ring 104, other devices may measure a user's temperature. In some examples, other wearable devices (e.g., wrist devices) may comprise sensors that measure user physiological parameters. Additionally, medical devices, such as external medical devices (e.g., wearable medical devices) and/or implantable medical devices, may measure a user's physiological parameters. One or more sensors on any type of computing device may be used to implement the techniques described herein.

The physiological measurements may be taken continuously throughout the day and/or night. In some implementations, the physiological measurements may be taken during portions of the day and/or portions of the night. In some implementations, the physiological measurements may be taken in response to determining that the user is in a specific state, such as an active state, a resting state, and/or a sleeping state. For example, the ring 104 may make physiological measurements in a resting/sleep state in order to acquire cleaner physiological signals. In one example, the ring 104 or other device/system may detect when a user is resting and/or sleeping and acquire physiological parameters (e.g., temperature) for that detected state. The devices/systems may use the resting/sleep physiological data and/or other data when the user is in other states in order to implement the techniques of the present disclosure.

In some implementations, as described previously herein, the ring 104 may be configured to collect, store, and/or process data, and may transfer any of the data described herein to the user device 106 for storage and/or processing. In some aspects of the invention, the user device 106 comprises a wearable application 250, an operating system (OS), a web browser application (e.g., web browser 280), one or more additional applications, and a GUI 275. The user device 106 may further comprise other modules and components, comprising sensors, audio devices, haptic feedback devices, and the like. The wearable application 250 may comprise an example of an application (e.g., “app”) that may be installed on the user device 106. The wearable application 250 may be configured to acquire data from the ring 104, store the acquired data, and process the acquired data as described herein. For example, the wearable application 250 may comprise a user interface (UI) module 255, an acquisition module 260, a processing module 230-b, a communication module 220-b, and a storage module (e.g., database 265) configured to store application data.

The various data processing operations described herein may be performed by the ring 104, the user device 106, the servers 110, or any combination thereof. For example, in some cases, data collected by the ring 104 may be pre-processed and transmitted to the user device 106. In this example, the user device 106 may perform some data processing operations on the received data, may transmit the data to the servers 110 for data processing, or both. For instance, in some cases, the user device 106 may perform processing operations that require relatively low processing power and/or operations that require a relatively low latency, whereas the user device 106 may transmit the data to the servers 110 for processing operations that require relatively high processing power and/or operations that may allow relatively higher latency.

In some aspects of the invention, the ring 104, user device 106, and server 110 of the system 200 may be configured to evaluate sleep patterns for a user. In particular, the respective components of the system 200 may be used to collect data from a user via the ring 104, and generate one or more scores (e.g., Sleep Score, Readiness Score) for the user based on the collected data. For example, as noted previously herein, the ring 104 of the system 200 may be worn by a user to collect data from the user, comprising temperature, heart rate, HRV, and the like. Data collected by the ring 104 may be used to determine when the user is asleep in order to evaluate the user's sleep for a given “sleep day.” In some aspects of the invention, scores may be calculated for the user for each respective sleep day, such that a first sleep day is associated with a first set of scores, and a second sleep day is associated with a second set of scores. Scores may be calculated for each respective sleep day based on data collected by the ring 104 during the respective sleep day. Scores may comprise, but are not limited to, Sleep Scores, Readiness Scores, and the like.

In some cases, “sleep days” may align with the traditional calendar days, such that a given sleep day runs from midnight to midnight of the respective calendar day. In other cases, sleep days may be offset relative to calendar days. For example, sleep days may run from 6:00 pm (18:00) of a calendar day until 6:00 pm (18:00) of the subsequent calendar day. In this example, 6:00 pm may serve as a “cut-off time,” where data collected from the user before 6:00 pm is counted for the current sleep day, and data collected from the user after 6:00 pm is counted for the subsequent sleep day. Due to the fact that most individuals sleep the most at night, offsetting sleep days relative to calendar days may enable the system 200 to evaluate sleep patterns for users in such a manner that is consistent with their sleep schedules. In some cases, users may be able to selectively adjust (e.g., via the GUI) a timing of sleep days relative to calendar days so that the sleep days are aligned with the duration of time that the respective users typically sleep.

In some implementations, each overall score for a user for each respective day (e.g., Sleep Score, Readiness Score) may be determined/calculated based on one or more “contributors,” “factors,” or “contributing factors.” For example, a user's overall Sleep Score may be calculated based on a set of contributors, comprising: total sleep, efficiency, restfulness, REM sleep, deep sleep, latency, timing, or any combination thereof. The Sleep Score may comprise any quantity of contributors. The “total sleep” contributor may refer to the sum of all sleep periods of the sleep day. The “efficiency” contributor may reflect the percentage of time spent asleep compared to time spent awake while in bed, and it may be calculated using the efficiency average of long sleep periods (e.g., primary sleep period) of the sleep day, weighted by a duration of each sleep period. The “restfulness” contributor may indicate how restful the user's sleep is, and it may be calculated using the average of all sleep periods of the sleep day, weighted by a duration of each period. The restfulness contributor may be based on a “wake up count” (e.g., sum of all the wake-ups (when user wakes up) detected during different sleep periods), excessive movement, and a “got up count” (e.g., sum of all the got-ups (when user gets out of bed) detected during the different sleep periods).

The “REM sleep” contributor may refer to a sum total of REM sleep durations across all sleep periods of the sleep day including REM sleep. Similarly, the “deep sleep” contributor may refer to a sum total of deep sleep durations across all sleep periods of the sleep day including deep sleep. The “latency” contributor may signify how long (e.g., average, median, longest) the user takes to go to sleep, and may be calculated using the average of long sleep periods throughout the sleep day, weighted by a duration of each period and the number of such periods (e.g., consolidation of a given sleep stage or sleep stages may be its own contributor or weight other contributors). Lastly, the “timing” contributor may refer to a relative timing of sleep periods within the sleep day and/or calendar day, and it may be calculated using the average of all sleep periods of the sleep day, weighted by a duration of each period.

By way of another example, a user's overall Readiness Score may be calculated based on a set of contributors, comprising: sleep, sleep balance, heart rate, HRV balance, recovery index, temperature, activity, activity balance, or any combination thereof. The Readiness Score may comprise any quantity of contributors. The “sleep” contributor may refer to the combined Sleep Score of all sleep periods within the sleep day. The “sleep balance” contributor may refer to a cumulative duration of all sleep periods within the sleep day. In particular, sleep balance may indicate to a user whether the sleep that the user has been getting over some duration of time (e.g., the past two weeks) is in balance with the user's needs. Typically, adults need 7-9 hours of sleep a night to stay healthy, alert, and to perform at their best both mentally and physically. However, it is normal to have an occasional night of bad sleep, so the sleep balance contributor takes into account long-term sleep patterns to determine whether each user's sleep needs are being met. The “resting heart rate” contributor may indicate a lowest heart rate from the longest sleep period of the sleep day (e.g., primary sleep period) and/or the lowest heart rate from naps occurring after the primary sleep period.

Continuing with reference to the “contributors” (e.g., factors, contributing factors) of the Readiness Score, the “HRV balance” contributor may indicate a highest HRV average from the primary sleep period and the naps happening after the primary sleep period. The HRV balance contributor may help users keep track of their recovery status by comparing their HRV trend over a first time period (e.g., two weeks) to an average HRV over some second, longer time period (e.g., three months). The “recovery index” contributor may be calculated based on the longest sleep period. Recovery index measures how long it takes for a user's resting heart rate to stabilize during the night. A sign of a very good recovery is that the user's resting heart rate stabilizes during the first half of the night, at least six hours before the user wakes up, leaving the body time to recover for the next day. The “body temperature” contributor may be calculated based on the longest sleep period (e.g., primary sleep period) or based on a nap happening after the longest sleep period if the user's highest temperature during the nap is at least 0.5° C. higher than the highest temperature during the longest period. In some aspects of the invention, the ring may measure a user's body temperature while the user is asleep, and the system 200 may display the user's average temperature relative to the user's baseline temperature. If a user's body temperature is outside of their normal range, the body temperature contributor may be highlighted (e.g., go to a “Pay attention” state) or otherwise generate an alert for the user.

Now proceeding into more specific embodiments discussing body heat generation model creation in more detail, FIGS. 3A-3G are also referred to.

Body temperature can be considered to be a sum or a combination of several heat generation and heat expulsion factors. One main part is the internal heat generation from the user body itself. This is balanced with external cooling factors caused by the environmental conditions, where the temperature of the user's surroundings acts in a major role.

Temperature sensors 240 have been described in the above paragraphs in many varying embodiments, but some further aspects are still discussed in the following concerning especially the presented method.

In an embodiment of a method according to the present invention, the starting point in the method is performing a temperature measurement with at least one temperature sensor positioned within a wearable device worn by the user. The desired number of temperature sensors are positioned within the housing of the wearable device. In an embodiment of the present invention, the wearable device is a ring having a group of temperature sensors positioned near the inner surface of the ring's housing. The temperature sensors may be positioned in equidistant mutual distances along (meaning: close to) the ring's inner circumferential edge, in an embodiment. However, in another embodiment, the temperature sensors may accommodate just a part of the complete inner circumference of the ring, meaning that the “first” temperature sensor and the “last” temperature sensor accommodate only a certain arc of the complete circular inner surface of the ring. In an embodiment, temperature sensor locations may be optimized, to have the best performance in this particular task. In practice, it is a compromise between different aspects which are part of the complete set of functionalities. The locations of the temperature sensors need to be well known for the design and algorithm to work properly. For example, when a good skin temperature is desired, the temperature sensors may be positioned close to the skin within the inner circumferential edge of the ring and when an environmental temperature is desired, then the temperature sensor(s) may be positioned close to the outside surface of the ring where the effect of the skin temperature on the sensor may be smaller and more accurate environmental temperature measurements may be enabled. Many variations in the temperature sensor locations are possible.

In an embodiment of the method according to the present invention, the system obtains 1 . . . N values of measured temperatures from different sides of the finger skin of the user, where N may be a natural number of 1, 2, . . . , 10, according to various applied embodiments of the present invention.

When the wearable device measuring the user skin temperatures is a wearable ring, in some embodiments the temperature sensors may face the finger skin directly, which in turn may enable the temperature measurement process of the user body beingless prone to effects originating from the surrounding space of the user (but not totally free of them). In an embodiment, the ring is provided with at least one specific functionality or property to help in obtaining this goal, e.g. one or more structural additions or a material selection enhancing insulation. This may enhance the accuracy of the temperature measurements.

Various cooling factors may affect directing the internal heat produced by the user body away from the skin surface of the user. Some of the cooling factors may depend on the movement status of the user; e.g. whether the user sits in an office at working hours, performs sports activities in the evening or sleeps in the bed at night. Also, environmental factors such as wind in the outdoors conditions, or draft possibly present indoors, may affect the cooling capabilities of the surrounding air in view of the finger and the worn ring.

In an embodiment of the present invention, one or more temperature values measured from the user are used in creating a model which estimates the internal heat generation produced in the tissues of the human finger and/or body. Heat generation and cooling can originate from a local source in the finger, or from a more distant source in the user's body. For instance, blood flow is able to carry heat from a longer distance within the body of the user, while sweating cools the skin and the tissues underneath locally. Environmental conditions are also taken into account in the model. Specific human body properties are part of the model, in an embodiment. These comprise heat convection information of the human tissues, involving soft tissues and bone tissue as well, in an embodiment. Device properties (e.g. wearable device properties or physical properties of the ring) are also part of the model, in an embodiment. Environmental parameter estimation may comprise temperature of the surrounding space of the user, movement/acceleration data of the measured finger over time, and for instance also humidity of the surrounding space (this affects the evaporation rate of the sweat present on the skin of the user, where the evaporation of sweat cools the respective skin surface). One option is to get information from a connected device, such as weather information from a respective weather app used in a smartphone.

In some embodiments of the present invention, a cross-sectional area or a relatively thin volume (e.g., “slice”) within the tissues of the user's finger may be modeled so that the user of the modelling tool is able to observe the thermal distribution i.e. different temperatures across the 2D or 3D tissue volume from the created model. When using a single temperature sensor, it is notable that the temperatures can be obtained only in 1D; then the system would estimate temperatures along a radial line from the center of the finger towards the sensor outside the finger. In other words, when applying at least two temperature sensors in the ring, the system may measure temperatures approximately along a circular line around the finger, and the task of the modelling is to determine the inner temperatures within the tissues of the finger, where the measuring ring is worn; also noting the environmental factors.

In the core of the model creation according to the invention, there is an inverse problem, where the temperatures are sensed along essentially a circumferential line, and the task is to model the temperatures inside this approximately circular line, by taking also other affecting factors and physical properties into account. The inverse problems generally relate to situations where there is a need to estimate parameters which cannot be directly measured.

From the obtained model, the user of the modelling tool is able to obtain useful information which relates to the wellbeing of the human user, stress levels of the user, data relating to metabolism of the user, possible sleep debt/freshness/readiness/other indication of the previous night's sleep qualitatively and/or quantitatively, certain responses to external stimuli or to internal changes such as taken medication or performed exercise, and even situations where the user is about to get sick, or is already mildly sick; in various embodiments of the present invention. In the last situation, the model could instruct the user to remain at home or in remote work, and thus, help to prevent infectious diseases from spreading to others so easily. Anything which could be sensed as an abnormal body temperature of the user, is here a useful result made possible by the presented method according to the invention. On the other hand, also the result which indicates that everything is alright within the user concerning health, stress level and freshness level (made possible after a good night's sleep), is valuable and useful.

Concerning a high-level solution of the present invention, the presented model may be based on geometrical simplification of a human finger (comprising skin and soft tissue) and a ring acting as a wearable device (comprising e.g. titanium and epoxy layers). In an embodiment, the temperature sensors of the ring are placed inside the epoxy layer. In an embodiment, it is assumed that constant heat generation is occurring inside the finger. In an embodiment, some method is needed to estimate the environment, which means that there is a variable boundary condition outside of the ring. The environment can be estimated based on the finger movement with an accelerometer, or with tagged activity, for instance. For example, heat convection can be notably different when the person is running compared to the situation when the person is sleeping.

In one embodiment of the model creation method according to the invention, the system applies a ring which has four temperature sensors inside the epoxy layer close to the inner surface of the cylindrical-shaped ring. In this presented example, the four temperature sensors are positioned in approximately 45 degree intervals along (or close to) the inner circumferential edge of the ring, and this situation is shown in FIGS. 3A and 3G as well. However, the temperature sensors can be located in other positions too, such as with same mutual intervals along the whole circumferential inner edge of the ring. One option is to have just a single temperature sensor, in an embodiment. Various numbers of temperature sensors may be applied in different embodiments of the invention.

In an embodiment of the invention, the model to be created estimates temperatures from the depth of 3 millimeters from the finger skin surface; thus, corresponding to approximative depth of the soft tissues of the human finger.

In an embodiment of the invention, the incoming heat flux is suggested to be produced in the human body comprising a human finger and the incoming heat flux is then propagated via the human tissues towards the temperature sensors of the ring. The outgoing heat flux is considered to comprise the outgoing thermal energy dissipating from the surface of the finger skin; near the interface area (i.e. near the contact area) between the ring and the finger.

In an embodiment of the invention, the outgoing heat flux may be determined based on a convection factor which describes the heat transfer from the human finger to surrounding air. The convection factor may be determined based on the movement of the user or based on the movement of the body part of the user, such as the hand. In turn, the movement of the user or the body part of the user may be tracked with one or more accelerometers within the housing of the ring, in an embodiment of the invention.

In an embodiment of the invention, the model to be created applies a finite difference method, where the incoming heat flux is considered to originate from the center point of the ring within the finger tissues. Heat conduction from the center point to each temperature sensor is one part of the model creation. The outgoing heat flux comprises the heat energy which propagates from the finger skin surface towards the surrounding volume of air. In an embodiment of the invention, the model may comprise other aspects which have an effect on the incoming and outgoing thermal energy flows.

In an embodiment of the invention, the presented method according to the invention solves both the outgoing and incoming heat fluxes, which fulfill the measured temperature values obtained in the group of temperature sensors within the ring.

In an embodiment of the invention, the system is designed to sense if the temperature values obtained by the group of temperature sensors are still fluctuating, concerning at least one temperature sensor. The system may be designed to sense when the temperature values of all the temperature sensors have stabilized so that the detected temperature values stay within certain threshold values.

Another phenomenon which affects the detected temperature values is noise within the measured temperature value behavior, i.e. small variations which can be seen as a “summed and rapidly varying signal” on top of the otherwise smooth temperature value behavior. In an embodiment of the invention, the noise can be cancelled at least partly by averaging the measured temperature values with a desired time window size.

If the resulting heat flows are known, they may be used to create a two-dimensional temperature map of the cross-section of the measured finger, or alternatively a 1D radial line as discussed earlier in connection with a single temperature sensor used in the ring. The temperature map may visualize the temperatures in each location of the cross-section by showing them with different colors.

Proceeding now to the following drawings, FIG. 3A shows an example of such a two-dimensional temperature result illustration of a cross-section of a user finger. FIG. 3A shows the cross-section of the tissues right in the place where the ring is worn, and the inner tissue temperatures are visualized in a lower section of the illustration in an approximately 135 degree arc within the soft tissues of the finger. The darker tone in this greyscale image means higher temperature. The temperature range is shown in the legend at the right-hand side of the illustration. The illustration shows also some exemplary incoming power and outgoing power values. The incoming heat fluxes towards each of the temperature sensors are shown with longer arrows starting from the center; and on the other hand, the shorter arrows positioned close to the housing of the ring are representing outgoing heat fluxes and their directions. The origin is in the center of the illustration, and the X- and Y-dimensions in view of the origin are shown in millimeters.

The illustration of FIG. 3A shows that while the inner tissues are naturally warmer than the outer tissues, there can be different cooling rates when looking at the finger in various outwards directions from the center of the finger.

FIGS. 3B-3D illustrate some exemplary results which show the measured temperature values from each applied temperature sensor, and some estimates of the inner and outer heat fluxes which are obtained from the created model.

FIG. 3B shows temperature values as a function of time for four temperature sensors, which are here named as “Sensor 1”, “Sensor 2”, “Sensor 3” and “Sensor 4”. These show the actual skin temperatures measured with the ring, in this example. The time scale in the horizontal axis is in seconds, where 30000 seconds correspond to 8 hours 20 minutes. It can be seen from FIG. 3B that at first the temperature values reach and stay in the range of 35.5 to 36.0 degrees Celsius, and changes in the finger situation can be seen simultaneously in all four sensors; although to a varying extent.

In around 10000 seconds time after the beginning, there is a dip in the temperatures, lasting for around 2000 seconds; the temperature values are then positioned between approximately 34 and 35° C. The biggest dip happens in around 18000 seconds' time. In general, this illustration shows the varying nature of the finger skin temperatures, when measuring during a night when the user is sleeping. It also shows that in different skin spots of the finger, the temperature slightly varies between the different sensor locations. In that sense, the human finger is not thermally fully “symmetrical.”

FIG. 3C illustrates an example of an inner heat flux, in W/m, determined with the help of the created model. The resulting inner heat flux values vary between 210 and 238 W/m. It can be seen that there is variety between the inner heat fluxes concerning the different temperature sensor locations. The “Sensor 2” gives the highest values, and “Sensor 4” gives the lowest values. Some variation as a function of time over the night, and also a sum signal regarded as noise can be seen especially with temperature sensors “Sensor 2”, “Sensor 1” and “Sensor 3”.

FIG. 3D illustrates an example of an outer heat flux, in W/m, determined with the help of the created model. The resulting outer heat flux values vary between −300 and +1000 W/m. Some variations can be seen as a function of time. What is notable, that all four temperature sensors experience the outer heat flux mutually almost similarly. Hence, the four graphs are positioned almost on top of one another.

In an embodiment of the invention, the method senses the noise present in the inner heat flux, for all used temperature sensors or a part of them, and then cancels the noise away from the inner heat flux values. This noise sensing may be performed by averaging the inner heat flux results over a certain window size, especially if it is noted that noise is both positive and negative over time on top of determined results. If the noise is a summed bias-type of value, then there needs to be some other method to study and cancel the noise effects from the determined results. Generally concerning noise properties, they can usually be seen as relatively quick changes in the measured temperature values, which are not possible in a real-world environment. The averaging of the noise-included measurement values of the temperature sensor smooths out at least a part of these rapid changes, and the result reflects the “real world” temperatures much more accurately.

FIGS. 3E, 3F and 3G illustrate more results, in another embodiment of the present invention.

FIG. 3E shows a temperature behavior graph, where at the start of wearing the ring and for instance going to bed, there can be seen a time period where the measured temperature value rapidly ascends and then settles in a more stable range of temperatures. The rapid ascent period can be seen between the vertical dashed lines at the start of the measurement period. Otherwise, during regular sleeping time and during constant wearing of the ring, the measured temperatures in this example vary between around 31.8° C.-35.5° C.

FIG. 3F illustrates an example of a manner in which the model according to the invention generates temperature values in the locations of each temperature sensor, as a function of tissue depth from the skin. In this example, there are seven temperature sensors: Sensor A, Sensor B, . . . , Sensor G. For instance, for “Sensor D” in the middle of the graph, the left-hand-most vertical bar shows a temperature value of appr. 33° C., which means that the measured temperature by “Sensor D” indeed equals this value. As the outer heat flux directs the thermal energy away from the tissue volume closer to the skin in an easier fashion, it means that the modelled temperature values stay relatively close to the value measured at the skin. However, the model shows that in the inner parts of the tissue, the temperature rises, as the incoming heat flux becomes more dominant, and the outgoing heat flux's effect diminishes. Hence, the inner temperature value obtained via “Sensor D”, and modelled in the innermost slice of the tissue, is close to 36° C. in this example. This is the darkest bar in the illustration, for each applied temperature sensor (i.e. the right-hand-most bar for each sensor).

FIG. 3G illustrates yet another example of a two-dimensional temperature result illustration of a cross-section of a user finger. This time the results are assembled from the modelled temperatures from FIG. 3F, but otherwise the illustrative manner is the same as in earlier FIG. 3A. This time the measured temperatures from the finger skin are notably lower than in the illustration of FIG. 3A. This means that the modelled inner temperatures are also lower compared to respective parts of the modelling according to FIG. 3A. The illustration of FIG. 3G also shows that the temperature value distribution across the whole cross-section is not necessarily symmetric in view of the origin of the illustration (i.e. the central point/axis of the finger).

It is also possible that some abnormal situation affects the measurements. If there is a gap between the finger and the ring, the temperature measurement may not be accurate. But depending on the location, in this case it might be possible to see from the optical signal that there is a contact problem. This can then be used to prevent false estimations.

It is emphasized that the number of applied temperature sensors and the presented graphs and temperature behavior within the finger tissues are merely exemplary. Many variations are possible. For instance, the number of temperature sensors may be just even one; although a better modelling result can be obtained with a plurality of temperature sensors placed in the ring.

As an advantage of the present invention, the implementation of the described method does not require any hardware changes. The presented method of the model creation comprises algorithmic, calculative steps and it requires processing the temperature measurements with the presented novel type of methodology.

As another implementation option of the present invention, specific heat flux sensors may be applied instead of the above discussed temperature sensors.

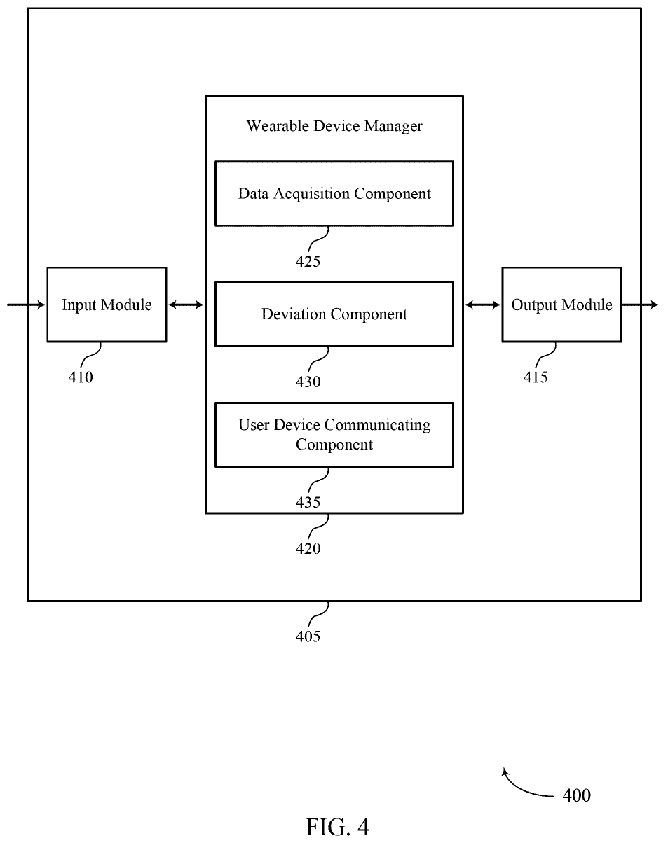

FIG. 4 illustrates a block diagram 400 of a device 405 that supports techniques for creating a body heat generation model of a user based on thermal measurements of the user by a wearable device in accordance with aspects of the present disclosure. The device 405 may include an input module 410, an output module 415, and a wearable device manager 420. The device 405 may also include a processor. Each of these components may be in communication with one another (e.g., via one or more buses).

For example, the wearable device manager 420 may include a data acquisition component 425, a deviation component 430, a user device communicating component 435, or any combination thereof. In some examples, the wearable device manager 420, or various components thereof, may be configured to perform various operations (e.g., receiving, monitoring, transmitting) using or otherwise in cooperation with the input module 410, the output module 415, or both. For example, the wearable device manager 420 may receive information from the input module 410, send information to the output module 415, or be integrated in combination with the input module 410, the output module 415, or both to receive information, transmit information, or perform various other operations as described herein.

The data acquisition component 425 may be configured as or otherwise support a means for acquiring physiological data associated with a physiological parameter of a user throughout a first time interval using a wearable device, wherein the physiological data is acquired according to a first periodicity based at least in part on one or more deviations in the physiological data failing to satisfy a deviation threshold. The deviation component 430 may be configured as or otherwise support a means for determining, using one or more processing components at the wearable device, that one or more additional deviations in the physiological data satisfy the deviation threshold. The data acquisition component 425 may be configured as or otherwise support a means for acquiring additional physiological data associated with the physiological parameter throughout a second time interval using the wearable device, wherein the additional physiological data is acquired according to a second periodicity that is greater than the first periodicity based at least in part on the one or more additional deviations satisfying the deviation threshold. The user device communicating component 435 may be configured as or otherwise support a means for transferring at least the additional physiological data from the wearable device to a user device associated with the wearable device based at least in part on establishing a wireless connection between the wearable device and the user device.

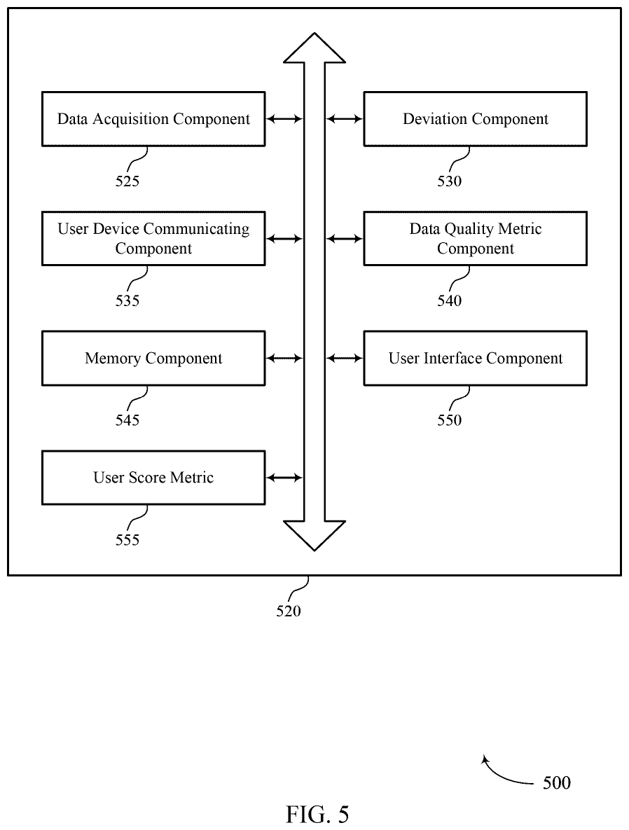

FIG. 5 illustrates a block diagram 500 of a wearable device manager 520 that supports techniques for creating a body heat generation model of a user based on thermal measurements of the user by a wearable device in accordance with aspects of the present disclosure. The wearable device manager 520 may be an example of aspects of a wearable device manager or a wearable device manager 420, or both, as described herein. The wearable device manager 520, or various components thereof, may be an example of means for performing various aspects of techniques for storing and transferring data collected by a wearable device as described herein. For example, the wearable device manager 520 may include a data acquisition component 525, a deviation component 530, a user device communicating component 535, a data quality metric component 540, a memory component 545, a user interface component 550, a user score metric 555, or any combination thereof. Each of these components may communicate, directly or indirectly, with one another (e.g., via one or more buses).

The data acquisition component 525 may be configured as or otherwise support a means for acquiring physiological data associated with a physiological parameter of a user throughout a first time interval using a wearable device, wherein the physiological data is acquired according to a first periodicity based at least in part on one or more deviations in the physiological data failing to satisfy a deviation threshold. The deviation component 530 may be configured as or otherwise support a means for determining, using one or more processing components at the wearable device, that one or more additional deviations in the physiological data satisfy the deviation threshold. In some examples, the data acquisition component 525 may be configured as or otherwise support a means for acquiring additional physiological data associated with the physiological parameter throughout a second time interval using the wearable device, wherein the additional physiological data is acquired according to a second periodicity that is greater than the first periodicity based at least in part on the one or more additional deviations satisfying the deviation threshold. The user device communicating component 535 may be configured as or otherwise support a means for transferring at least the additional physiological data from the wearable device to a user device associated with the wearable device based at least in part on establishing a wireless connection between the wearable device and the user device.

In some examples, the data quality metric component 540 may be configured as or otherwise support a means for determining one or more data quality metrics associated with the physiological data, the one or more data quality metrics associated with a relative quality or accuracy of the physiological data. In some examples, the memory component 545 may be configured as or otherwise support a means for discarding the one or more data quality metrics based at least in part on the one or more deviations in the physiological data failing to satisfy the deviation threshold.

In some examples, the data quality metric component 540 may be configured as or otherwise support a means for determining one or more additional data quality metrics associated with the additional physiological data, the one or more additional data quality metrics associated with a relative quality or accuracy of the additional physiological data. In some examples, the memory component 545 may be configured as or otherwise support a means for storing the one or more additional data quality metrics in a memory at the wearable device. In some examples, the user device communicating component 535 may be configured as or otherwise support a means for transferring the one or more additional data quality metrics to the user device based at least in part on storing the one or more additional data quality metrics in the memory.

In some examples, the user score metric 555 may be configured as or otherwise support a means for updating a physiological metric, a score, or both, associated with the physiological parameter based at least in part on the additional physiological data and the one or more additional data quality metrics, and based at least in part on transferring the additional physiological data and the one or more additional data quality metrics to the user device. In some examples, the user interface component 550 may be configured as or otherwise support a means for causing a GUI of the user device to display information associated with the physiological metric, the score, or both, based at least in part on the updating.

In some examples, the memory component 545 may be configured as or otherwise support a means for storing the physiological data in a memory at the wearable device based at least in part on the first periodicity. In some examples, the memory component 545 may be configured as or otherwise support a means for storing the additional physiological data in the memory based at least in part on the second periodicity. In some examples, the user device communicating component 535 may be configured as or otherwise support a means for transferring the physiological data and the additional physiological data to the user device based at least in part on storing the physiological data and the additional physiological data in the memory.

In some examples, the user device communicating component 535 may be configured as or otherwise support a means for transmitting, from the wearable device to the user device, an indication that the one or more deviations in the physiological data fail to satisfy the deviation threshold.

In some examples, the deviation component 530 may be configured as or otherwise support a means for determining, after an expiration of the second time interval, that a third set of one or more deviations in the additional physiological data fail to satisfy the deviation threshold. In some examples, the data acquisition component 525 may be configured as or otherwise support a means for acquiring third physiological data of the user via the wearable device throughout a third time interval, wherein the third physiological data is acquired according to the first periodicity based at least in part on the third set of one or more deviations in the additional physiological data failing to satisfy the deviation threshold.

In some examples, the data acquisition component 525 may be configured as or otherwise support a means for determining a duration of the second time interval based at least in part on the physiological data acquired using the wearable device.

In some examples, the deviation threshold is based at least in part on the physiological parameter. In some examples, the physiological parameter comprises a blood oxygen saturation metric, a bioimpedance metric, or both.

In some examples, the physiological data is further associated with an additional physiological parameter, and the data acquisition component 525 may be configured as or otherwise support a means for acquiring the physiological data associated with the additional physiological parameter using the wearable device throughout the first time interval according to the second periodicity, a third periodicity, or both.

In some examples, the physiological data associated with the additional physiological parameter is acquired according to the second periodicity, the third periodicity, or both, regardless of whether deviations in the physiological data associated with the additional physiological parameter satisfy the deviation threshold, an additional deviation threshold, or both. In some examples, the physiological parameter comprises a blood oxygen saturation metric. In some examples, the additional physiological parameter comprises a heart rate metric, an HRV metric, or both.

In some examples, the user interface component 550 may be configured as or otherwise support a means for causing a GUI of the user device to display information associated with the physiological data, the additional physiological data, or both. In some examples, the wearable device comprises a wearable ring device.

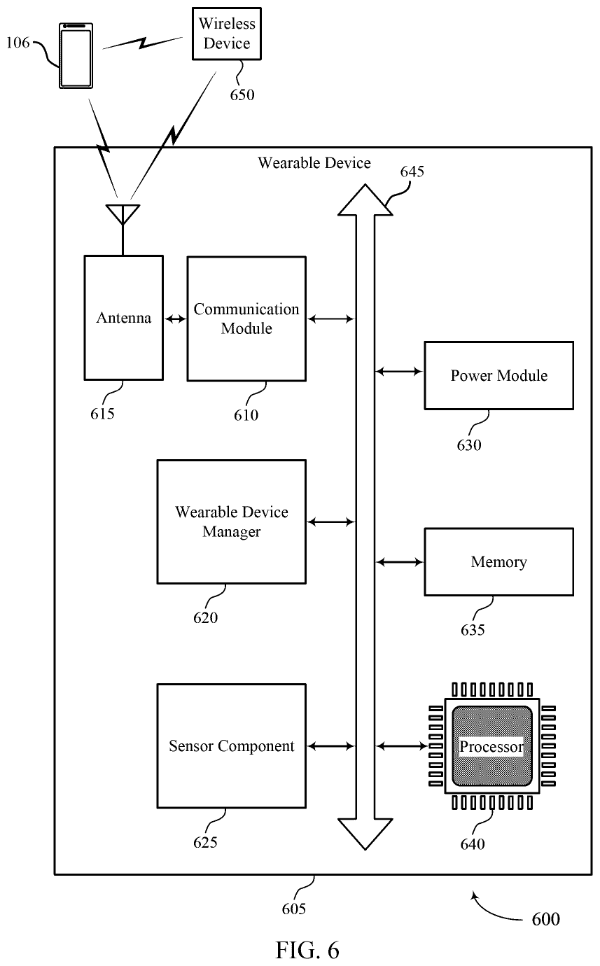

FIG. 6 illustrates a diagram of a system 600 comprising a device 605 that supports techniques for creating a body heat generation model of a user based on thermal measurements of the user by a wearable device in accordance with aspects of the present disclosure. The device 605 may be an example of or comprise the components of a device 405 as described herein. The device 605 may comprise an example of a wearable device 104, as described previously herein. The device 605 may comprise components for bi-directional communications comprising components for transmitting and receiving communications with a user device 106 and a server 110, such as a wearable device manager 620, a communication module 610, an antenna 615, a sensor component 625, a power module 630, a memory 635, a processor 640, and a wireless device 650. These components may be in electronic communication or otherwise coupled (e.g., operatively, communicatively, functionally, electronically, electrically) via one or more buses (e.g., a bus 645).

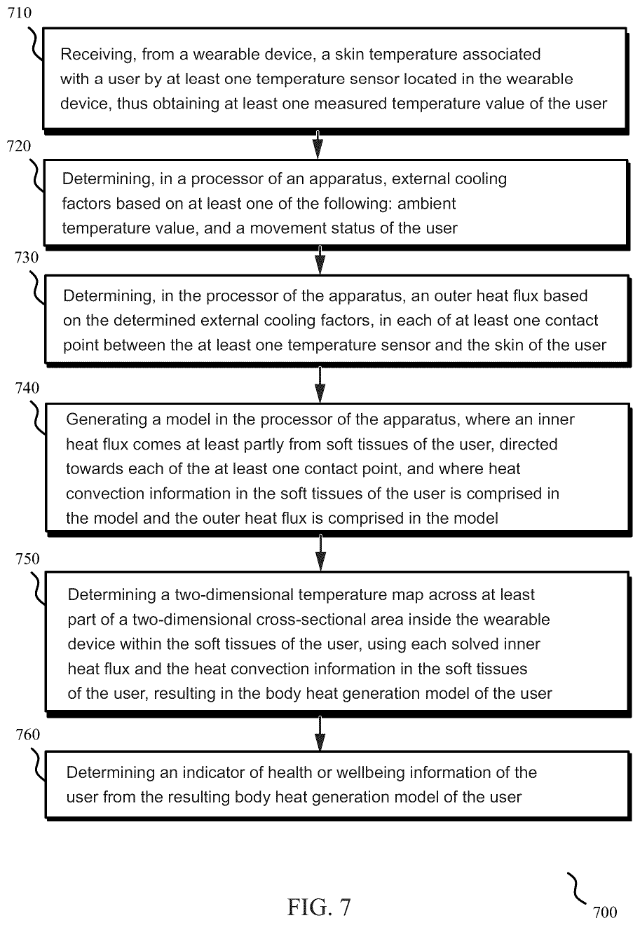

FIG. 7 illustrates a flowchart showing a method 700 that supports techniques for creating a body heat generation model of a user based on thermal measurements of the user by a wearable device in accordance with aspects of the present disclosure. The operations of the method 700 may be implemented by a wearable device or its components as described herein. For example, the operations of the method 700 may be performed by a wearable device as described with reference to FIGS. 1 through 6. In some examples, a wearable device may execute a set of instructions to control the functional elements of the wearable device to perform the described functions. Additionally, or alternatively, the wearable device may perform aspects of the described functions using special-purpose hardware.

At step 710, the method may comprise receiving, from a wearable device, a skin temperature associated with a user by at least one temperature sensor located in the wearable device, thus obtaining at least one measured temperature value of the user. The operations of 710 may be performed in accordance with examples as disclosed herein.

At step 720, the method may comprise determining, in a processor of an apparatus, external cooling factors based on at least one of the following: ambient temperature value, and a movement status of the user. The operations of 720 may be performed in accordance with examples as disclosed herein.

At step 730, the method may comprise determining, in the processor of the apparatus, an outer heat flux based on the determined external cooling factors, in each of at least one contact point between the at least one temperature sensor and the skin of the user. The operations of 730 may be performed in accordance with examples as disclosed herein.

At step 740, the method may comprise generating a model in the processor of the apparatus, where an inner heat flux comes at least partly from soft tissues of the user, directed towards each of the at least one contact point, and where heat convection information in the soft tissues of the user is comprised in the model and the outer heat flux is comprised in the model. The operations of 740 may be performed in accordance with examples as disclosed herein.

At step 750, the method may comprise determining a two-dimensional temperature map across at least part of a two-dimensional cross-sectional area inside the wearable device within the soft tissues of the user, using each solved inner heat flux and the heat convection information in the soft tissues of the user, resulting in the body heat generation model of the user. The operations of 750 may be performed in accordance with examples as disclosed herein.

At step 760, the method may comprise determining an indicator of health or wellbeing information of the user from the resulting body heat generation model of the user. The operations of 760 may be performed in accordance with examples as disclosed herein.

Discussing possible application areas and result types in various embodiments of the present invention, the following is referred to.

From the determined indicator of health or wellbeing information of the user, in practice it is possible to sense e.g. something concerning metabolism of the user, or something related to certain early phases of catching a disease, such as Covid-19, for instance. An early suspicion or prediction of a Covid-19 infection could be seen through some change in the generated body heat generation model, and it is possible to combine the invented method with some other method, like an official test or an at-home test, to enhance the reliability of the test result. One advantage is the result being obtained earlier, which decreases the risks of spreading the disease to others. One possible application area is inspecting a reaction to some sort of change the user is experiencing, like as a response to taken medication, or as a response to a tougher training period with athletes. General stress levels (or general “recovery”/or not slept well the previous night) experienced by the user can be seen with the presented method according to the invention, as described also earlier in the description.

It should be noted that the methods described above describe possible implementations, and that the operations and the steps may be rearranged or otherwise modified and that other implementations are possible. Furthermore, aspects from two or more of the methods may be combined.

A method is described. The method may comprise receiving, from a wearable device, a skin temperature associated with a user by at least one temperature sensor located in the wearable device, thus obtaining at least one measured temperature value of the user; determining, in a processor of an apparatus, external cooling factors based on at least one of the following: ambient temperature value, and a movement status of the user; determining, in the processor of the apparatus, an outer heat flux based on the determined external cooling factors, in each of at least one contact point between the at least one temperature sensor and the skin of the user; generating a model in the processor of the apparatus, where an inner heat flux comes at least partly from soft tissues of the user, directed towards each of the at least one contact point, and where heat convection information in the soft tissues of the user is comprised in the model and the outer heat flux is comprised in the model; determining a two-dimensional temperature map across at least part of a two-dimensional cross-sectional area inside the wearable device within the soft tissues of the user, using each solved inner heat flux and the heat convection information in the soft tissues of the user, resulting in the body heat generation model of the user; and determining an indicator of health or wellbeing information of the user from the resulting body heat generation model of the user.

An apparatus is described. The apparatus may comprise a processor, a memory coupled with the processor, and instructions stored in the memory. The instructions may be executable by the processor to cause the apparatus to receive, from a wearable device, a skin temperature associated with a user by at least one temperature sensor located in the wearable device, thus obtaining at least one measured temperature value of the user; determine external cooling factors based on at least one of the following: ambient temperature value, and a movement status of the user; determine an outer heat flux based on the determined external cooling factors, in each of at least one contact point between the at least one temperature sensor and the skin of the user; generate a model, where an inner heat flux comes at least partly from soft tissues of the user, directed towards each of the at least one contact point, and where heat convection information in the soft tissues of the user is comprised in the model and the outer heat flux is comprised in the model; determine a two-dimensional temperature map across at least part of a two-dimensional cross-sectional area inside the wearable device within the soft tissues of the user, using each solved inner heat flux and the heat convection information in the soft tissues of the user, resulting in the body heat generation model of the user; and determine an indicator of health or wellbeing information of the user from the resulting body heat generation model of the user.

Another apparatus is described. The apparatus may comprise means for receiving, from a wearable device, a skin temperature associated with a user by at least one temperature sensor located in the wearable device, thus obtaining at least one measured temperature value of the user; means for determining external cooling factors based on at least one of the following: ambient temperature value, and a movement status of the user; means for determining an outer heat flux based on the determined external cooling factors, in each of at least one contact point between the at least one temperature sensor and the skin of the user; means for generating a model, where an inner heat flux comes at least partly from soft tissues of the user, directed towards each of the at least one contact point, and where heat convection information in the soft tissues of the user is comprised in the model and the outer heat flux is comprised in the model; means for determining a two-dimensional temperature map across at least part of a two-dimensional cross-sectional area inside the wearable device within the soft tissues of the user, using each solved inner heat flux and the heat convection information in the soft tissues of the user, resulting in the body heat generation model of the user; and means for determining an indicator of health or wellbeing information of the user from the resulting body heat generation model of the user.

A non-transitory computer-readable medium storing code is described. The code may comprise instructions executable by a processor to receive, from a wearable device, a skin temperature associated with a user by at least one temperature sensor located in the wearable device, thus obtaining at least one measured temperature value of the user; determine external cooling factors based on at least one of the following: ambient temperature value, and a movement status of the user; determine an outer heat flux based on the determined external cooling factors, in each of at least one contact point between the at least one temperature sensor and the skin of the user; generate a model, where an inner heat flux comes at least partly from soft tissues of the user, directed towards each of the at least one contact point, and where heat convection information in the soft tissues of the user is comprised in the model and the outer heat flux is comprised in the model; determine a two-dimensional temperature map across at least part of a two-dimensional cross-sectional area inside the wearable device within the soft tissues of the user, using each solved inner heat flux and the heat convection information in the soft tissues of the user, resulting in the body heat generation model of the user; and determine an indicator of health or wellbeing information of the user from the resulting body heat generation model of the user.

Some examples of the method, apparatuses, and non-transitory computer-readable medium described herein may further include operations, features, means, or instructions for sensing movement of the user or a part of the body of the user by the wearable device using at least one accelerometer; determining the movement status of the user; and using the movement status of the user and data obtained from an external user device in determining the external cooling factors.

Some examples of the method, apparatuses, and non-transitory computer-readable medium described herein may further include operations, features, means, or instructions for using heat flux sensors in the place of the at least one temperature sensor.

Some examples of the method, apparatuses, and non-transitory computer-readable medium described herein may further include operations, features, means, or instructions for using the body heat generation model for creating a health or wellbeing status parameter (or e.g. a status indicator) of the user, comprising illness detection, illness prediction, stress level indication, revival of metabolism, menopause determination/prediction based on hot flashes of female users, and period prediction of female users, as examples among many possible options.

Some examples of the method, apparatuses, and non-transitory computer-readable medium described herein may further include operations, features, means, or instructions for using four temperature sensors in the wearable device for the skin temperature measurements.

Some examples of the method, apparatuses, and non-transitory computer-readable medium described herein may further include operations, features, means, or instructions, wherein the wearable device is a ring.

Some examples of the method, apparatuses, and non-transitory computer-readable medium described herein may further include operations, features, means, or instructions for using at least two temperature sensors in the ring for the skin temperature measurements; and for estimating temperature values in a depth of the soft tissue from the surface of the finger skin in a soft tissue volume of the user, thus forming the two-dimensional temperature map.

Some examples of the method, apparatuses, and non-transitory computer-readable medium described herein may further include operations, features, means, or instructions for, if a detected skin temperature measurement result differs from an expected value by more than a predetermined threshold value, informing the user in an external user device that the ring has either a wrong size for the user or the ring is not in a proper contact with the finger skin of the user.

Some examples of the method, apparatuses, and non-transitory computer-readable medium described herein may further include operations, features, means, or instructions for detecting noise in the inner and/or the outer heat flux; and canceling the detected noise from the inner and/or the outer heat flux, before calculating the body heat generation model of the user.

Some examples of the method, apparatuses, and non-transitory computer-readable medium described herein may further include operations, features, means, or instructions for performing the calculation of the body heat generation model of the user only after the measured skin temperatures of the user have stabilized to stay within set threshold limits.

Where an external user device is discussed, it may for instance be a smartphone or a tablet or a personal computer of the user wearing the wearable device, such as a ring.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples”. The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a DSP, an ASIC, an FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration).

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described above can be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on”.

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media can comprise RAM, ROM, electrically erasable programmable ROM (EEPROM), compact disk (CD) ROM or other optical disk storage, magnetic disk storage or other magnetic storage devices, or any other non-transitory medium that can be used to carry or store desired program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein but is to be accorded the broadest scope consistent with the principles and novel features disclosed herein.

1. A method for creating a body heat generation model of a user, comprising:
receiving, from a wearable device, a skin temperature associated with a user by at least one temperature sensor located in the wearable device, thus obtaining at least one measured temperature value of the user;
determining, in a processor of an apparatus, external cooling factors based on at least one of the following: ambient temperature value, and a movement status of the user;
determining, in the processor of the apparatus, an outer heat flux based on the determined external cooling factors, in each of at least one contact point between the at least one temperature sensor and the skin of the user;
generating a model in the processor of the apparatus, where an inner heat flux comes at least partly from soft tissues of the user, directed towards each of the at least one contact point, and wherein the model comprises heat convection information in the soft tissues of the user and the outer heat flux;
determining a two-dimensional temperature map across at least part of a two-dimensional cross-sectional area inside the wearable device within the soft tissues of the user, using each solved inner heat flux and the heat convection information in the soft tissues of the user, resulting in the body heat generation model of the user; and
determining an indicator of health or wellbeing information of the user from the resulting body heat generation model of the user.

2. The method according to claim 1, wherein the method further comprises the steps of:
sensing movement of the user or a part of the body of the user by the wearable device using at least one accelerometer;
determining the movement status of the user; and
using the movement status of the user and data obtained from an external user device in determining the external cooling factors.

3. The method according to claim 1, wherein the method further comprises a step of:
using heat flux sensors for the at least one temperature sensor.

4. The method according to claim 1, wherein the method further comprises a step of:
using the body heat generation model for creating a health or wellbeing status parameter of the user, comprising illness detection, illness prediction, stress level indication, revival of metabolism, menopause determination/prediction based on hot flashes of female users, and period prediction of female users.

5. The method according to claim 1, wherein the method further comprises a step of:
using four temperature sensors in the wearable device for the skin temperature measurements.

6. The method according to claim 1, wherein the wearable device is a ring.

7. The method according to claim 6, wherein the method further comprises steps of:
using at least two temperature sensors in the ring for the skin temperature measurements; and
estimating temperature values in a depth of the soft tissue from the surface of the finger skin in a soft tissue volume of the user, and forming the two-dimensional temperature map.

8. The method according to claim 6, wherein if a detected skin temperature measurement result differs from an expected value by more than a predetermined threshold value, the method further comprises a step of: informing the user in an external user device that the ring has either a wrong size for the user or the ring is not in a proper contact with the finger skin of the user.

9. The method according to claim 1, wherein the method further comprises the steps of:
detecting noise in the inner and/or the outer heat flux; and
canceling the detected noise from the inner and/or the outer heat flux, before calculating the body heat generation model of the user.

10. The method according to claim 1, wherein the method further comprises a step of:
performing the calculation of the body heat generation model of the user only after the measured skin temperatures of the user have stabilized to stay within set threshold limits.

11. An apparatus for creating a body heat generation model of a user, the apparatus comprising:
a processor;
a memory coupled with the processor, and instructions stored in the memory; the instructions being executable by the processor to cause the apparatus to:
receive, from a wearable device, a skin temperature associated with a user by at least one temperature sensor located in the wearable device, thus obtaining at least one measured temperature value of the user;
determine external cooling factors based on at least one of the following: ambient temperature value, and a movement status of the user;
determine an outer heat flux based on the determined external cooling factors, in each of at least one contact point between the at least one temperature sensor and the skin of the user;
generate a model, wherein an inner heat flux comes at least partly from soft tissues of the user, directed towards each of the at least one contact point, and wherein the model comprises heat convection information in the soft tissues of the user and the outer heat flux;
determine a two-dimensional temperature map across at least part of a two-dimensional cross-sectional area inside the wearable device within the soft tissues of the user, using each solved inner heat flux and the heat convection information in the soft tissues of the user, resulting in the body heat generation model of the user; and
determine an indicator of health or wellbeing information of the user from the resulting body heat generation model of the user.

12. An apparatus for creating a body heat generation model of a user, the apparatus comprising:
means for receiving, from a wearable device, a skin temperature associated with a user by at least one temperature sensor located in the wearable device, thus obtaining at least one measured temperature value of the user;
means for determining external cooling factors based on at least one of the following: ambient temperature value, and a movement status of the user;
means for determining an outer heat flux based on the determined external cooling factors, in each of at least one contact point between the at least one temperature sensor and the skin of the user;
means for generating a model, wherein an inner heat flux comes at least partly from soft tissues of the user, directed towards each of the at least one contact point, and wherein the model comprises heat convection information in the soft tissues of the user and the outer heat flux;
means for determining a two-dimensional temperature map across at least part of a two-dimensional cross-sectional area inside the wearable device within the soft tissues of the user, using each solved inner heat flux and the heat convection information in the soft tissues of the user, resulting in the body heat generation model of the user; and
means for determining an indicator of health or wellbeing information of the user from the resulting body heat generation model of the user.

13. A non-transitory computer-readable medium for creating a body heat generation model of a user, the computer-readable medium storing code, which code comprises instructions executable by a processor to:
receive, from a wearable device, a skin temperature associated with a user by at least one temperature sensor located in the wearable device, thus obtaining at least one measured temperature value of the user;
determine external cooling factors based on at least one of the following: ambient temperature value, and a movement status of the user;
determine an outer heat flux based on the determined external cooling factors, in each of at least one contact point between the at least one temperature sensor and the skin of the user;
generate a model, where an inner heat flux comes at least partly from soft tissues of the user, directed towards each of the at least one contact point, and where heat convection information in the soft tissues of the user is comprised in the model and the outer heat flux is comprised in the model;
determine a two-dimensional temperature map across at least part of a two-dimensional cross-sectional area inside the wearable device within the soft tissues of the user, using each solved inner heat flux and the heat convection information in the soft tissues of the user, resulting in the body heat generation model of the user; and
determine an indicator of health or wellbeing information of the user from the resulting body heat generation model of the user.
